(19) United States
(12) Patent Application Publication
(10) Pub. No.: US 2009/0053140 A1
(43) Pub. Date: Feb. 26, 2009

(54) METHODS OF IDENTIFYING GENES INVOLVED IN MEMORY FORMATION USING SMALL INTERFERING RNA(siRNA)

(76) Inventors: Roderick Euan Milne Scott, Poway, CA (US); Rusiko Bourtchouladze, New York, NY (US); Marco Peters, La Jolla, CA (US); Timothy P. Tully, Solana Beach, CA (US)

Correspondence Address: DON J. PELTO;Sheppard, Mullin, Richter & Hampton LLP, 1300 I STREET, NW, 11TH FLOOR EAST, WASHINGTON, DC, 20005, US

(21) Appl. No.: 12/121,742

(22) Filed: May 15, 2008

Related U.S. Application Data
(60) Provisional application No. 60/938,165, filed on May 15, 2007.

(51) Int. Cl. A61K 49/00

(52) U.S. Cl. 424/9.1

(57) ABSTRACT

The present invention relates to a method of identifying a gene or gene product associated with transcription dependent memory formation in an animal comprising the steps of: (a) administering to said animal sufficient small interfering RNA (siRNA) specific for the gene to inhibit gene function; (b) training said animal under conditions sufficient to induce transcription dependent memory formation in a normal untreated animal; and (c) determining the level of transcription dependent memory formation induced by the training of the treated animal. The present invention provides methods of using small interfering PNAs (siRNA) in hippocampus to identify genes and gene product whose inhibition affects contextual and temporal long-term (LTM) memory, but not short-term memory (STM).

### RELATED APPLICATIONS

This application claims the benefit, under 35 U.S.C. §119, of provisional U.S. Application Ser. No. 60/938,165, filed May 15, 2007, the entire contents and substance of which is hereby incorporated by reference.

### FIELD OF THE INVENTION

The present invention relates to methods of identifying genes involved in memory formation using small interfering RNA (siRNA) molecules.

### BACKGROUND OF THE INVENTION

An attribute that many organisms, including humans, possess is memory of past events. This attribute has been studied for many decades with much information now available that explains many of its ramifications. For example, two basic types of memory have been identified: transcription-independent memory, which includes short term memory, and transcription-dependent memory, which includes long term memory.

The identification of genes associated with memory formation would provide (a) a genetic epidemiology of cognitive dysfunction, (b) diagnostic tools for individuals carrying different allelic forms of these genes (associative with different performance levels for particular forms of cognition) and (c) new targets for drug discovery ultimately to ameliorate various forms of cognitive dysfunction (and particular drugs could be matched to particular forms of cognitive dysfunction by the diagnostic tests). Thus, it would be useful to have techniques available that would identify the genes that are associated with memory formation.

A relatively unknown aspect of memory is the identity of genes that contribute to its manifestation. A method for the identification of genes that may contribute to memory formation is described in U.S. Pat. No. 7,005,256 through the use of differential screen to identify additional “downstream” genes that are transcriptionally regulated during transcription-dependent memory formation. DNA probes were synthesized using RNA extracted from the heads of spaced- or massed-trained flies according to methods generally known in the art. RNA was extracted from fly heads. Spaced- and massed-training of flies were conducted as described previously. Complementary DNA (cDNA) probes were synthesized from the extracted RNA. The complex cDNA probe mixture then was hybridized onto microarray chips containing DNA sequences. The signal from hybridized DNA probes was amplified and detected. A statistical comparison was performed by comparing the signal detected between spaced- and massed-trained groups to identify candidate genes.

However, there is a need for a method to test the candidate genes to confirm that such genes are transcriptionally regulated during transcription-dependent memory formation.

RNA interference (RNAi) provides a new gene-silencing technique to investigate the biological mechanisms of gene function and has potential for in vivo target validation. RNAi by synthetic 21-nucleotide small interfering RNA douplexes (siRNA) have been used to study gene-function in cultured cells (Elbashir et al., 2001, Nature 411:494-498). However, successful delivery of synthetic siRNA to the CNS in vivo have been limited by the low efficiency of naked siRNA, therefore requiring the use of large amounts of siRNA or the expression of siRNA from viral vectors (Thakker et al., 2004, Proc. Natl. Acad Sci USA 101:17270-17275); (Xia et al., 2002, Nat. Biotechnol. 20:1006-1010). Furthermore, specific effects of RNAi on memory formation have not been demonstrated so far.

Both contextual and trace conditioning require the function of intact hippocampus (Phillips and LeDoux, 1992, Behav. neurosci 1006:274-285); (McEchron et al., 1998, Hippocampus 8:638-646). In contextual conditioning, a previously neutral context is paired with a mild, unavoidable foot-shock. In trace conditioning, a short interval (a trace) is imposed between a conditioned stimulus such as tone (CS) and unconditioned stimulus such as a shock (US). This short interval increases the complexity of the learning task sufficiently as to require the hippocampus (Kim et al., 1995, Behav. Neurosci. 109:195-203); (McGlinchey-Berroth et al., 1997, Behav Neursci 111:973-882); (Clark and Squire, 1998, Science 280:77-81); (McEchron et al., 1998, Hippocampus 8:638-646); (Buchel et al., 1999, J. Neurosci 19:10869-10876). As such, trace conditioning bears resemblance to contextual conditioning in which an animal does not simply associate a conditioned stimulus with an unconditioned stimulus, but associates the conditioned stimulus with the whole context in which they are exposed to the conditioned stimulus.

There is a need to identify genes and protein products associated with the development of contextual and temporal long-term memory in the hippocampus.

### SUMMARY OF THE INVENTION

The present invention is related to the discovery that siRNA of candidate genes can be used to determine the effect of the inhibition of candidate genes involved in transcription-dependent memory formation, particularly long term memory formation.

Particularly, in one embodiment the present invention includes a method comprising the steps of: (a) administering to an animal sufficient siRNA specific for a gene to inhibit the gene's function; (b) training the animal under conditions sufficient to induce transcription dependent memory formation in a normal untreated animal; and (c) determining the level of transcription dependent memory formation induced by the training of the treated animal.

In another embodiment the determination of an increase in transcription dependent memory formation in the treated animal relative to the transcription dependent memory formation in an untreated animal indicates that inhibition of the gene results in enhancement of transcription dependent memory formation. In another embodiment the determination of a decrease in transcription dependent memory formation in the treated animal relative to the transcription dependent memory formation in an untreated animal indicates that inhibition of the gene results in inhibition of transcription dependent memory formation.

In a particular embodiment, the transcription dependent memory formation is long term memory formation. In another embodiment the transcription dependent memory formation is evidenced by performance of a specific cognitive task.

Another embodiment of the present invention includes a method comprising the steps of: (a) administering to an animal sufficient siRNA specific for a gene to inhibit the gene's function; (b) training the animal under conditions sufficient to induce long term memory formation in a normal untreated animal; and (c) determining the level of long term memory formation induced by the training of the treated animal.

In one embodiment the determination of an increase in long term memory formation in the treated animal relative to the long term memory formation in an untreated animal indicates that inhibition of the gene results in enhancement of long term memory formation. In another embodiment the determination of a decrease in long term memory formation in the treated animal relative to the long term memory formation in an untreated animal indicates that inhibition of the gene results in inhibition of long term memory formation.

In a particular embodiment, the long term memory formation is evidenced by performance of a specific cognitive task.

Another embodiment of the present invention includes a method comprising the steps of: (a) administering to an animal sufficient siRNA specific for a gene to inhibit the gene's function; (b) training the animal under conditions sufficient to produce an improvement in performance of a specific cognitive task in a normal untreated animal; and (c) determining the level of cognitive performance generated by training of the treated animal.

In one embodiment the determination of the level of cognitive performance in the treated animal relative to the level of cognitive performance in an untreated animal indicates that inhibition of the gene results in enhancement of cognitive performance. In another embodiment, the determination of a decrease in the level of cognitive performance in the treated animal relative to the level of cognitive performance in an untreated animal indicates that inhibition of the gene results in inhibition of cognitive performance.

In a particular embodiment the cognitive performance is long term memory formation. In another embodiment the cognitive performance is evidenced by performance of a specific cognitive task.

In all embodiments, the siRNA can be administered before or simultaneously with the training session. In all embodiments, the animal can be a non-human mammal. In all embodiments, the step (b) training can comprise multiple training sessions. In all embodiments, the step (b) training can comprise a spaced training protocol. In all embodiments, the step (b) training can comprise a contextual fear training protocol with single or multiple trials. In all embodiments, the step (b) training can comprise trace fear conditioning with single or multiple trials. In all embodiments, the training can relate to a memory paradigm selected from the group consisting of contextual memory, temporal memory, spatial memory, episodic memory, passive avoidance memory, active avoidance memory, social transmission of food preferences memory, conditioned taste avoidance, and social recognition memory.

These and other aspects of the invention will become evident upon reference to the following detailed description and attached drawings. It is to be understood however that various changes, alterations and substitutions may be made to the specific embodiments disclosed herein without departing from their essential spirit and scope. In addition, it is further understood that the drawings are intended to be illustrative and symbolic representations of an exemplary embodiment of the present invention and that other non-illustrated embodiments are within the scope of the invention.

### BRIEF DESCRIPTION OF THE DRAWINGS

FIG. 1a. is a bar graph showing in Neuro2A cells of CREB mRNA, PP1 [symbol] mRNA, the NMDA receptor subunit 1 (Grin1) mRNA and Synaptotagmin I (Syt1) mRNA after treatment with CREB siRNA. The mean±stdev of two to four experimental replications are shown. Open bars: vehicle, stripped bars: non-targeting, grey bars: CREB1 siRNA; black bars: CREB2 siRNA.

FIG. 1b. is a bar graph showing the level in Neuro2A cells of CREB mRNA, PP1 [symbol] mRNA, the NMDA receptor subunit 1 (Grin 1) mRNA and Synaptotagmin I (Syt 1) mRNA after treatment with PP1\acute{α} siRNA. The mean±stdev of two experimental replications are shown. Open bars: vehicle, stripped bars: non-targeting, grey bars: PP1\acute{α} siRNA

FIG. 2a. is a photograph of the coronal sections of hippocampus injected with Cy3 labeled siRNA and 22 kDa polyethyleneimine carrier.

FIG. 2b. is a Western Blot of hippocampal levels of CREB protein and Synaptotagmin protein in mice after injection of non-targeting (scrambled) siRNA or CREB siRNA injection. FIG. 2b also shows a bar graph showing the level of hippocampal CREB protein and Synaptotagmin protein in mice after injection of non-targeting (scrambled) siRNA or CREB1 siRNA injection.

FIG. 2c is a bar graph showing the percentage of context freezing of mice during training (immediate freezing), 30 minutes after training (short term memory) and 24 hours after training (long term memory) after injection of non-targeting (scrambled) siRNA or CREB siRNA injection.

FIG. 2d. a bar graph showing the percentage of freezing of mice during training (immediate freezing), 30 minutes after training (short term memory) and 24 hours after training (long term memory) after injection of non-targeting (scrambled) siRNA or CREB siRNA.

FIG. 3a. is a bar graph showing the percentage of contextual freezing in C57BL/6 mice during training, 30 minutes after training and 24 hours after training after injection of non-targeting (scrambled) siRNA or CREB siRNA2.

FIG. 3b is a schematic diagram of a training protocol for post-training siRNA infusions.

FIG. 3c. is a bar graph showing the percentage of contextual freezing in C57BL/6 mice during training and 7 days after training after injection of non-targeting (scrambled) siRNA or CREB siRNA2 by the protocol shown in FIG. 3b.

FIG. 4a. is a Western Blot showing the level of PP1\acute{α} and CREB protein in the hippocampus after PP1\acute{α} siRNA injection. FIG. 4a is also a bar graph of the level of PP1α and CREB protein in the hippocampus after PP1\acute{α} siRNA injection

FIG. 4b. is a bar graph showing the percent of context freezing in C57BL/6 mice during training and 24 hours after training after injection of non-targeting (scrambled) siRNA or PP1\acute{α} siRNA.

FIG. 4c. is a bar graph showing the percent freezing in C57BL/6 mice during training, 24 hours after training pre conditioned stimulus and 24 hours after training and upon tone conditioned stimulus.

FIG. 5a. is a bar graph showing the effect of number of training trials on contextual memory formation. Mice were trained with increasing numbers of CS-US pairings and contextual memory assessed 4 days later.

FIG. 5b is a bar graph showing the effect of the trace interval on temporal memory formation. Mice were trained in trace fear conditioning using increasingly long trace intervals and tone memory compared to delay conditioning.

FIG. 6a is a table of the level of mRNA expression within mouse CNS as measured by real-time PCR.

FIG. 6b is a table of the level of mRNA expression within mouse CNS as measured by real-time PCR.

FIG. 7 is a bar graph of the mRNA levels of Gpr12 24 hours after siRNA treatment in Neuro2A cells.

FIG. 8a is a bar graph of the effect of Gpr12 siRNA in mouse hippocampus on contextual memory.

FIG. 8b is a bar graph of the effect of Gpr12 siRNA in mouse amygdala on contextual memory.

FIG. 9 is a bar graph of the effect of Gpr12 siRNA in mouse hippocampus on trace fear memory.

FIG. 10 is a picture of Nissl stain of non-targeting (A) and Gpr12 siRNA (B) on infused hippocampus. Hippocampal slices of the dorsal and ventral of the cannula insertion site are shown.

FIG. 11 is a bar graph of the hippocampal Gpr12 mRNA levels 2 and 3 days after Gpr12 siRNA treatment.

### DETAILED DESCRIPTION OF THE INVENTION

The present invention is related to the discovery that siRNA of candidate genes can be used to identify and characterize the effect of inhibition of candidate genes involved in transcription-dependent memory formation, particularly long term memory formation.

Transcription-independent memory includes various “memory phases”, such as short-term memory, intermediate-(or middle-)term memory and (in flies) anesthesia-resistant memory. In common to these forms is that pharmacological inhibitors of RNA transcription do not disrupt these memories. Transcription-dependent memory usually is referred to as long-term memory and inhibitors of RNA synthesis block its appearance.

The invention is directed to a method of identifying a gene or gene product associated with transcription dependent memory formation in a non-human animal comprising the steps of: (a) administering to said animal sufficient siRNA specific for the gene to inhibit gene function; (b) training said animal under conditions sufficient to induce transcription dependent memory formation in a normal untreated animal; and (c) determining the level of transcription dependent memory formation induced by the training of the treated animal.

To produce a specific “long-term memory,” an animal is subjected to a specific training protocol under controlled, experimental conditions. In Pavlovian conditioning procedures, for instance, two specific stimuli are presented in temporal contiguity to produce “associative learning and memory.” One of the two stimuli is designated a “conditioned stimulus” (CS) and the other is designated an “unconditioned stimulus” (US). The US usually is a natural re-enforcer that elicits a “unconditioned response” (UR) before training in a “reflexive” manner. With CS-US pairing, a “conditioned response” (CR) begins to appear in response to the CS before (or in the absence of) presentation of the US. After a CR to a specific CS-US pairing is “learned”, memory formation thereafter begins.

Memory formation of this specific, experimental experience can exist in two general forms: a transcription-independent form and a transcription-dependent form. The former includes various “memory phases,” such as short-term memory, and intermediate-(or middle) term memory. In common to these forms is that pharmacological inhibitors of RNA transcription do not disrupt these memories. The latter form usually is referred to as long-term memory and inhibitors of RNA synthesis block its appearance.

In animal models, various experimental treatments, such as gene mutation, pharmacological blockade, anatomical lesion or specific training protocols, can affect one or more of these types of memories. In particular, some experimental treatments yield normal amounts of transcription-independent memory but do not yield transcription-dependent memory. Such observations constitute the basis of informative DNA chip comparisons. In general, a comparison is made between two experimental protocols; one (experimental group) that is sufficient to induce both transcription-independent and transcription-dependent memories and one that yields only transcription-independent memory (control group). Any detectable differences in transcript levels between these two protocols then can be attributed specifically to a transcription-dependent memory of the experimentally induced learning. These transcripts are referred to herein as “Candidate Memory Genes” (CMGs).

Although experimental conditions are controlled to induce a specific type of learning, other experimentally uncontrolled forms of learning also may take place. Thus, although a control group may not yield transcription-dependent memory of the specific experimental task, it nevertheless may yield a transcription-dependent memory of an uncontrolled learning experience. One type of such experience is the potential “nonassociative” forms of learning that occur in response to only the CS or US (alone), or in response to CS-US presentations that are not paired temporally (which is the key requirement for “associative learning”). Hence, transcription-dependent “nonspecific” memories may exist in control groups, as defined above. This observation gives rise to a broader class of transcripts involved with “nonspecific” learning, which we refer to as Candidate Plasticity Genes (CPGs). DNA chip comparisons between an experimental group, as defined above, and naive (untrained) animals will yield CPGs, along with CMGs.

Behavior-genetic studies in Drosophila have established a pair of training protocols with differential effects on memory formation after a Pavlovian odor-shock learning paradigm. Ten training sessions “massed” together (i.e., with no rest interval between sessions) yields maximal learning (acquisition) and transcription-independent memories (not protein synthesis-dependent) (early memories, short-term memory). In contrast, ten training sessions “spaced” (i.e., with a 15-minute rest interval between sessions) yields equivalent levels of learning and transcription-independent memories (early memories), as well as maximal levels of transcription-dependent memory (including protein synthesis-dependent long-term memory (LTM)). LTM requires spaced training; even 48 massed training sessions fails to induce LTM (Tully et al., Cell, 79:35 47 (1994)). Protein synthesis-dependent LTM induced by spaced training is blocked completely via overexpression of CREB repressor (Yin et al., Cell, 79:49 58 (1994)). The resulting memory curve after spaced training, where protein synthesis- and CREB-dependent LTM is blocked, is similar to that produced by massed training in normal flies. In contrast, overexpression of CREB activator induces LTM with less training (one training session) or with massed training (Yin et al., Cell, 81:107 115 (1995)). Hence, the induction of LTM is both protein synthesis- and CREB-dependent. These results demonstrate that the only functional difference between spaced and massed training protocols is the appearance of transcription-dependent memory after the former.

The statistical procedures described above only suggest “statistical candidates.” A fundamental aspect of the statistical methods employed (as well as other such methods) is that “false positive” and “false negative” candidates are obtained along with the “true positives.” Hence, an independent method of detecting experience-dependent changes in gene transcription must be applied to the “statistical candidates.”

Most genes in mice have been shown to have human homologs. With the growing knowledge that human homologs can be functionally substituted in mice for its mouse homolog, the present discovery directly implicates the corresponding human homologs.

The differential effects on long-lasting memory produced by spaced versus massed training is a phenomenon widely observed in the animal kingdom. In particular, a spaced-massed differential effect on long-lasting memory recently has been established for the conditioned fear-potentiated startle effect in rats (a mammalian model system). In the fear-potentiated startle paradigm, memory is inferred from an increase in startle amplitude in the presence of a conditioned stimulus (CS) that has been previously paired with footshock. Massed training in rats (4-CS-shock pairings with a 10-second intertrial interval) produces essentially no transcription-dependent memory whereas spaced training (4 pairings with an 8-minute intertrial interval) produces significant transcription-dependent memory. (Josselyn et al., Society for Neurosci., 24: 926, Abstract 365.10 (1998)).

Unless defined otherwise, technical and scientific terms used herein have the same meaning as commonly understood by one of skill in the art to which this invention belongs.

One skilled in the art will recognize many methods and materials similar to equivalent to those described here, which could be used in the practice of this invention. Indeed the present invention is no way limited to the methods and materials described herein. For the purposes of the present invention, the following terms are defined.

### DEFINITIONS

The term “animal”, as used herein, includes mammals, as well as other animals, vertebrate and invertebrate (e.g., birds, fish, reptiles, insects (e.g., Drosophila species), Aplysia). The terms “mammal” and “mammalian”, as used herein, refer to any vertebrate animal, including monotremes, marsupials and placental, that suckle their young and either give birth to living young (eutharian or placental mammals) or are egg-laying (metatharian or nonplacental mammals). Examples of mammalian species include humans and other primates (e.g., monkeys, chimpanzees), rodents (e.g., rats, mice, guinea pigs) and ruminents (e.g., cows, pigs, horses). The methods of the invention will be used with non-human mammals.

As used herein, a “control animal” or a “normal animal” is an animal that is of the same species as, and otherwise comparable to (e.g., similar age, sex), the animal that is trained under conditions sufficient to induce transcription-dependent memory formation in that animal.

By “modulate” is meant that the expression of the gene, or level of RNA molecule or equivalent RNA molecules encoding one or more proteins or protein subunits, or activity of one or more proteins or protein subunits is up regulated or down regulated, such that expression, level, or activity is greater than or less than that observed in the absence of the modulator. For example, the term “modulate” can mean “inhibit,” but the use of the word “modulate” is not limited to this definition.

By “inhibit”, “down-regulate”, or “reduce”, it is meant that the expression of the gene, or level of RNA molecules or equivalent RNA molecules encoding one or more proteins or protein subunits, or activity of one or more proteins or protein subunits, is reduced below that observed in the absence of the nucleic acid molecules (e.g., siNA) of the invention. In one embodiment, inhibition, down-regulation or reduction with an siNA molecule is below that level observed in the presence of an inactive or attenuated siRNA molecule. In another embodiment, inhibition, down-regulation, or reduction with siNA molecules is below that level observed in the presence of, for example, an siNA molecule with scrambled sequence or with mismatches. In another embodiment, inhibition, down-regulation or reduction with an siRNA molecule is meant that the expression level of the target RNA molecules or equivalent RNA molecules is reduced by at least 20%, 30%, 40%, 50%, 60%, or 70% compared to the level in the absence of the siRNA molecules.

By “enhancing” or “enhancement” is meant the ability to potentiate, increase, improve or make greater or better, relative to normal, a biochemical or physiological action or effect. For example, enhancing long term memory formation refers to the ability to potentiate or increase long term memory formation in an animal relative to the normal long term memory formation of the animal. As a result, long term memory acquisition is faster or better retained. Enhancing performance of a cognitive task refers to the ability to potentiate or improve performance of a specified cognitive task by an animal relative to the normal performance of the cognitive task by the animal.

The term “candidate memory gene” or “target gene” or gene” means, a nucleic acid that encodes an RNA, for example, nucleic acid sequences including, but not limited to, structural genes encoding a polypeptide. The target gene can be a gene derived from a cell or an endogenous gene. By “target nucleic acid” is meant any nucleic acid sequence whose expression or activity is to be modulated. The target nucleic acid can be DNA or RNA.

By “homologous sequence” is meant, a nucleotide sequence that is shared by one or more polynucleotide sequences, such as genes, gene transcripts and/or non-coding polynucleotides. For example, a homologous sequence can be a nucleotide sequence that is shared by two or more genes encoding related but different proteins, such as different members of a gene family, different protein epitopes, different protein isoforms or completely divergent genes, such as a cytokine and its corresponding receptors. A homologous sequence can be a nucleotide sequence that is shared by two or more non-coding polynucleotides, such as noncoding DNA or RNA, regulatory sequences, introns, and sites of transcriptional control or regulation. Homologous sequences can also include conserved sequence regions shared by more than one polynucleotide sequence. Homology does not need to be perfect homology (e.g., 100%), as partially homologous sequences are also contemplated by the instant invention (e.g., 99%, 98%, 97%, 96%, 95%, 94%, 93%, 92%, 91%, 90%, 89%, 88%, 87%, 86%, 85%, 84%, 83%, 82%, 81%, 80% etc.).

By “conserved sequence region” is meant, a nucleotide sequence of one or more regions in a polynucleotide does not vary significantly between generations or from one biological system, subject, or organism to another biological system, subject, or organism. The polynucleotide can include both coding and non-coding DNA and RNA.

By “sense region” is meant a nucleotide sequence of a siNA molecule having complementarity to an antisense region of the siNA molecule. In addition, the sense region of a siNA molecule can comprise a nucleic acid sequence having homology with a target nucleic acid sequence.

By “antisense region” is meant a nucleotide sequence of a siNA molecule having complementarity to a target nucleic acid sequence. In addition, the antisense region of a siNA molecule can optionally comprise a nucleic acid sequence having complementarity to a sense region of the siNA molecule.

By “complementarity” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al., 1987, CSH Symp. Quant. Biol. LII pp. 123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonucleotide being based paired to a second nucleic acid sequence having 10 nucleotides represents 50%, 60%, 70%, 80%, 90%, and 100% complementary respectively). “Perfectly complementary” means that all the contiguous residues of a nucleic acid sequence will hydrogen bond with the same number of contiguous residues in a second nucleic acid sequence.

By “RNA” is meant a molecule comprising at least one ribonucleotide residue. By “ribonucleotide” is meant a nucleotide with a hydroxyl group at the 2′ position of a β-D-ribofuranose moiety. The terms include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siNA or internally, for example at one or more nucleotides of the RNA. Nucleotides in the RNA molecules of the instant invention can also comprise non-standard nucleotides, such as non-naturally occurring nucleotides or chemically synthesized nucleotides or deoxynucleotides. These altered RNAs can be referred to as analogs or analogs of naturally-occurring RNA.

The term “phosphorothioate” as used herein refers to an internucleotide linkage n an RNA molecule wherein at least one linkage between two nucleotides comprises a sulfur atom. Hence, the term phosphorothioate refers to both phosphorothioate and phosphorodithioate internucleotide linkages.

The term “phosphonoacetate linkage” as used herein refers to an internucleotide linkage in an RNA molecule wherein at least one linkage between two nucleotides comprises an acetyl or protected acetyl group. See for example Sheehan et al., 2003 Nucleic Acids Research 31, 4109-4118 or U.S. Patent Publication No. 2006/0247194

The term “thiophosphonoacetate linkage” as used herein refers to an RNA molecule comprising at least one internucleotide linkage comprising an acetyl or protected acetyl group and a sulfur atom. See for example Sheehan et al., 2003 Nucleic Acids Research 31, 4109-4118 or U.S. Patent Publication No. 2006/0247194.

### Identification of Candidate Genes

Candidate genes for the present invention can be initially identified by a number of means. A method for the identification of genes that may contribute to memory formation is described in U.S. Pat. No. 7,005,256 through the use of differential screen to identify additional “downstream” genes that are transcriptionally regulated during transcription-dependent memory formation. The animals were trained under conditions necessary to elicit transcription dependent memory formation. RNA was extracted from brain tissue (such as from amydala, hippocampus) of the trained animals. DNA probes were synthesized using the extracted and the DNA probes were contacted with microarray chips containing DNA sequences from genes of the genome of the animals under conditions appropriate for hybridization of the DNA probes to complementary DNA sequences on the microarray chips. A statistical comparison between the signal detected from RNA produced during transcription dependent memory formation compared to RNA produced during transcription independent memory formation was conducted to identify the candidate memory genes.

### Training Protocols

In various species, long-term memory (LTM) is defined by two main biological properties. First, formation of long-term memory requires synthesis of new proteins. Second, it involves cAMP-responsive transcription and is mediated through the cAMP-response element binding protein (CREB) family transcription factors.

Transcription-dependent memory can be induced using specific experimental conditions. In one embodiment, transcription-dependent memory is induced in a non-human animal using a spaced training protocol for the fear-potentiated startle response. In a second embodiment, transcription-dependent memory is induced in a non-human animal using a shuttle-box avoidance protocol. In a third embodiment, transcription-dependent memory is induced in a non-human animal using a contextual fear conditioning protocol.

Contextual fear conditions is a form of associative learning in which animals learn to recognize a training environment (conditioned stimulus, CS) that has been previously paired with an aversive stimulus such as foot shock (unconditioned stimulus, US). When exposed to the same context at a later time, conditioned animals show a variety of conditional fear responses, including freezing behavior (Fanselow, M. S., Behav. Neurosci., 98:269-277 (1984); Fanselow, M. S., Behav. Neurosci., 98:79-95 (1984); and Phillips, R. G. and LeDoux, J. E., Behav. Neurosci., 106:274-285 (1992)). Contextual conditioning has been used to investigate the neural substrates mediating fear-motivated learning (Phillips, R. G. and LeDoux, J. E., Behav. Neurosci., 106:274-285 (1992); and Kim, J. J. et al., Behav. Neurosci., 107:1093-1098 (1993)). Recent studies in mice and rats provided evidence for functional interaction between hippocampal and nonhippocampal systems during contextual conditioning training (Maren, S. et al., Behav. Brain Res., 88(2):261-274 (1997); Maren, S. et al., Neurobiol. Learn. Mem., 67(2):142-149 (1997); and Frankland, P. W. et al., Behav. Neurosci., 112:863-874 (1998)). Specifically, post-training lesions of the hippocampus (but not pre-training lesions) greatly reduced contextual fear, implying that: 1) the hippocampus is essential for contextual memory but not for contextual learning per se and 2) in the absence of the hippocampus during training, non-hippocampal systems can support contextual conditioning.

Contextual conditioning has been extensively used to study the impact of various mutations on hippocampus-dependent learning and memory (Bourtchouladze et al., Cell, 79:59-68 (1994); Bourtchouladze et al., Learn Mem., 5(4-5):365-374 (1998); Kogan, J. H. et al., Current Biology, 7(1):1-11 (1997); Silva A. J. et al., Current Biology, 6(11):1509-1518 (1996); Abel, T. et al., Cell, 88:615-626 (1997); and Giese, K. P. et al., Science, 279:870-873 (1998)) and strain differences in mice (Logue, S. F. et al., Neuroscience, 80(4):1075-1086 (1997); Chen, C. et al., Behav. Neurosci., 110:1177-1180 (1996); and Nguyen, P. V. et al., Learn Mem., 7(3): 170-179 (2000)). Because robust learning can be triggered with a few minutes training session, contextual conditioning has been especially useful to study the biology of temporally distinct processes of short- and long-term memory (Kim, J. J. et al., Behav. Neurosci., 107:1093-1098 (1993); Abel, T. et al., Cell, 88:615-626 (1997); Bourtchouladze et al., Cell, 79:59-68 (1994); Bourtchouladze et al., Learn Mem., 5(4-5):365-374 (1998)). As such, contextual conditioning provides an excellent model to evaluate the role of various novel genes in hippocampal-dependent memory formation.

Other training protocols can also be used in accordance with the present invention as will be understood by those of ordinary skill in the art. These training protocols can be directed towards the evaluation of, without limitation, hippocampus and/or amygdala dependent memory formation or cognitive performance. Non-limiting examples of additional appropriate training protocols include those that incorporate and/or relate to multiple training sessions, spaced training sessions, contextual fear training with single or multiple trials, trace fear conditioning with single or multiple trials, contextual memory generally, temporal memory, spatial memory, episodic memory, passive avoidance memory, active avoidance memory, social transmission of food preferences memory, conditioned taste avoidance, and/or social recognition memory.

### RNA Molecules

Once a target sequence or sequences have been identified in accordance with the invention, the appropriate siRNA can be produced, for example, either synthetically or by expression in cells. In a one embodiment, the DNA sequences encoding the antisense strand of the siRNA molecule can be generated by PCR. In another embodiment, the siRNA encoding DNA is cloned into a vector, such as a plasmid or viral vector, to facilitate transfer into mammals. In another embodiment, siRNA molecules may be synthesized using chemical or enzymatic means.

In one embodiment of the present invention, each sequence of a siNA molecules of the invention is independently about 18 to about 30 nucleotides in length, in specific embodiments about 18, 19, 20, 21, 22, 23, 24, 25, 26, 27, 28, 29 or 30 nucleotides in length. In one embodiment, the siRNA molecules contain about 19-23 base pairs, and preferably about 21 base pairs. In another embodiment, the siRNA molecules contain about 24-28 base pairs, and preferably about 26 base pairs. Individual siRNA molecules may be in the form of single strands, as well as paired double strands (“sense” and “antisense”) and may include secondary structure such as a hairpin loop. Individual siRNA molecules could also be delivered as precursor molecules, which are subsequently altered to give rise to active molecules. Examples of siRNA molecules in the form of single strands include a single stranded anti-sense siRNA against a non-transcribed region of a DNA sequence (e.g. a promoter region). In yet another embodiment, siNA molecules of the invention comprising hairpin or circular structures are about 35 to about 55 (e.g., about 35, 40, 45, 50 or 55) nucleotides in length, or about 38 to about 44 (e.g., about 38, 39, 40, 41, 42, 43, or 44) nucleotides in length and comprising about 16 to about 22 (e.g., about 16, 17, 18, 19, 20, 21 or 22) base pairs.

The discussion that follows discusses the proposed mechanism of RNA interference mediated by short interfering RNA as is presently known, and is not meant to be limiting and is not an admission of prior art. Chemically-modified short interfering nucleic acids possess similar or improved capacity to mediate RNAi as do siRNA molecules and are expected to possess improved stability and activity in vivo. Therefore, this discussion is not meant to be limiting only to siRNA and can be applied to siNA as a whole.

RNA interference refers to the process of sequence specific post-transcriptional gene silencing in animals mediated by short interfering RNAs (siRNAs) (Fire et al., 1998, Nature, 391, 806). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes which is commonly shared by diverse flora and phyla (Fire et al., 1999, Trends Genet., 15, 358). Such protection from foreign gene expression may have evolved in response to the production of double-stranded RNAs (dsRNAs) derived from viral infection or the random integration of transposon elements into a host genome via a cellular response that specifically destroys homologous single-stranded RNA or viral genomic RNA. The presence of dsRNA in cells triggers the RNAi response though a mechanism that has yet to be fully characterized. This mechanism appears to be different from the interferon response that results from dsRNA-mediated activation of protein kinase PKR and 2′,5′-oligoadenylate synthetase resulting in non-specific cleavage of mRNA by ribonuclease L.

The presence of long dsRNAs in cells stimulates the activity of a ribonuclease III enzyme referred to as Dicer. Dicer is involved in the processing of the dsRNA into short pieces of dsRNA known as short interfering RNAs (siRNAs) (Berstein et al., 2001, Nature, 409, 363). Short interfering RNAs derived from Dicer activity are typically about 21 to about 23 nucleotides in length and comprise about 19 base pair duplexes. Dicer has also been implicated in the excision of 21- and 22-nucleotide small temporal RNAs (stRNAs) from precursor RNA of conserved structure that are implicated in translational control (Hutvagner et al., 2001, Science, 293, 834). The RNAi response also features an endonuclease complex containing a siRNA, commonly referred to as an RNA-induced silencing complex (RISC), which mediates cleavage of single-stranded RNA having sequence homologous to the siRNA. Cleavage of the target RNA takes place in the middle of the region complementary to the guide sequence of the siRNA duplex (Elbashir et al., 2001, Genes Dev., 15, 188). In addition, RNA interference can also involve small RNA (e.g., micro-RNA or mRNA) mediated gene silencing, presumably though cellular mechanisms that regulate chromatin structure and thereby prevent transcription of target gene sequences (see for example Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237).

RNAi has been studied in a variety of systems. Fire et al., 1998, Nature, 391, 806, were the first to observe RNAi in C. elegans. Wianny and Goetz, 1999, Nature Cell Biol., 2, 70, describe RNAi mediated by dsRNA in mouse embryos. Hammond et al., 2000, Nature, 404, 293, describe RNAi in Drosophila cells transfected with dsRNA. Elbashir et al., 2001, Nature, 411, 494, describe RNAi induced by introduction of duplexes of synthetic 21-nucleotide RNAs in cultured mammalian cells including human embryonic kidney and HeLa cells. Recent work in Drosophila embryonic lysates has revealed certain requirements for siRNA length, structure, chemical composition, and sequence that are essential to mediate efficient RNAi activity. These studies have shown that 21 nucleotide siRNA duplexes are most active when containing two 2-nucleotide 3′-terminal nucleotide overhangs. Furthermore, substitution of one or both siRNA strands with 2′-deoxy or 2′-O-methyl nucleotides abolishes RNAi activity, whereas substitution of 3′-terminal siRNA nucleotides with deoxy nucleotides was shown to be tolerated. Mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end (Elbashir et al., 2001, EMBO J., 20, 6877). Other studies have indicated that a 5′-phosphate on the target-complementary strand of a siRNA duplex is required for siRNA activity and that ATP is utilized to maintain the 5′-phosphate moiety on the siRNA (Nykanen et al., 2001, Cell, 107, 309); however, siRNA molecules lacking a 5′-phosphate are active when introduced exogenously, suggesting that 5′-phosphorylation of siRNA constructs may occur in vivo.

In one embodiment, the invention features modified siNA molecules. Examples of modifications contemplated for the phosphate backbone include phosphate backbone modifications comprising one or more phosphorothioate, phosphorodithioate, phosphonates, including methylphosphonate, phosphotriester including alkylphosphotriesters, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications, see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39.

Examples of modifications contemplated for the sugar moiety include 2′-alkyl pyrimidine, such as 2′-O-methyl, 2′-fluoro, amino, and deoxy modifications and the like (see, e.g., Amarzguioui et al., 2003, Nucleic Acids Res. 31:589-595. U.S. Patent Publication No. 2007/0104688). Examples of modifications contemplated for the base groups include abasic sugars, 2-O-alkyl modified pyrimidines, 4-thiouracil, 5-bromouracil, 5-iodouracil, and 5-(3-aminoallyl)-uracil and the like. Locked nucleic acids, or LNA's, could also be incorporated. Many other modifications are known and can be used so long as the above criteria are satisfied. Examples of modifications are also disclosed in U.S. Pat. Nos. 5,684,143, 5,858,988 and 6,291,438 and in U.S. published patent application No. 2004/0203145 A1, each incorporated herein by reference. Other modifications are disclosed in Herdewijn (2000), Antisense Nucleic Acid Drug Dev. 10:297-310, Eckstein (2000) Antisense Nucleic Acid Drug Dev. 10:117-21, Rusckowski et al. (2000) Antisense Nucleic Acid Drug Dev. 10:333-345, Stein et al. (2001) Antisense Nucleic Acid Drug Dev. 11:317-25 and Vorobjev et al. (2001) Antisense Nucleic Acid Drug Dev. 11:77-85, each incorporated herein by reference

RNA may be produced enzymatically or by partial/total organic synthesis, and modified ribonucleotides can be introduced by in vitro enzymatic or organic synthesis. In one embodiment, each strand is prepared chemically. Methods of synthesizing RNA molecules are known in the art.

Other methods that can be used in accordance with the present invention include but are not limited to homologous recombination, transgenic expression of dominant-negative gene constructs, transgenic expression of normal gene constructs and any other modification of amino acid sequence in the target gene. Viral vectors can also be used to deliver various such gene constructs to brain cells; such constructs include several which act via the RNAi pathway (short hairpin RNA, double stranded RNA, etc).

### Formulations

The siRNA sample can be suitably formulated and introduced into the environment of the cell by any means that allows for a sufficient portion of the sample to enter the cell to induce gene silencing, if it is to occur. Many formulations for dsRNA are known in the art and can be used so long as siRNA gains entry to the target cells so that it can act. See, e.g., U.S. published patent application Nos. 2004/0203145 A1 and 2005/0054598 A1, each incorporated herein by reference. For example, siRNA can be formulated in buffer solutions such as phosphate buffered saline solutions, liposomes, micellar structures, and capsids. Formulations of siRNA with cationic lipids can be used to facilitate transfection of the dsRNA into cells. For example, cationic lipids, such as lipofectin (U.S. Pat. No. 5,705,188, incorporated herein by reference), cationic glycerol derivatives, and polycationic molecules, such as polylysine (published PCT International Application WO 97/30731, incorporated herein by reference), can be used. Suitable lipids include Oligofectamine, Lipofectamine (Life Technologies), NC388 (Ribozyme Pharmaceuticals, Inc., Boulder, Colo.), or FuGene 6 (Roche) all of which can be used according to the manufacturer's instructions.

In one embodiment, siNA molecules of the invention are formulated or complexed with polyethylenimine (e.g., linear or branched PEI) and/or polyethylenimine derivatives, including for example grafted PEIs such as galactose PEI, cholesterol PEI, antibody derivatized PEI, and polyethylene glycol PEI (PEG-PEI) derivatives thereof (see for example Ogris et al., 2001, AAPA PharmSci, 3, 1-11; Furgeson et al., 2003, Bioconjugate Chem., 14, 840-847; Kunath et al., 2002, Pharmaceutical Research, 19, 810-817; Choi et al., 2001, Bull. Korean Chem. Soc., 22, 46-52; Bettinger et al., 1999, Bioconjugate Chem., 10, 558-561; Peterson et al., 2002, Bioconjugate Chem., 13, 845-854; Erbacher et al., 1999, Journal of Gene Medicine Preprint, 1, 1-18; Godbey et al., 1999, PNAS USA, 96, 5177-5181; Godbey et al., 1999, Journal of Controlled Release, 60, 149-160; Diebold et al., 1999, Journal of Biological Chemistry, 274, 19087-19094; Thomas and Klibanov, 2002, PNAS USA, 99, 14640-14645; and Sagara, U.S. Pat. No. 6,586,524, incorporated by reference herein.

It can be appreciated that the method of introducing siRNA into the environment of the cell will depend on the type of cell and the make up of its environment. For example, when the cells are found within a liquid, one preferable formulation is with a lipid formulation such as in lipofectamine and the siRNA can be added directly to the liquid environment of the cells. Lipid formulations can also be administered to animals such as by intravenous, intramuscular, or intraperitoneal injection, or orally or by inhalation or other methods as are known in the art. When the formulation is suitable for administration into animals such as mammals and more specifically humans, the formulation is also pharmaceutically acceptable. Pharmaceutically acceptable formulations for administering oligonucleotides are known and can be used. In some instances, it may be preferable to formulate siRNA in a buffer or saline solution and directly inject the formulated dsRNA into cells. The direct injection of dsRNA duplexes may also be done. For suitable methods of introducing siRNA see U.S. published patent application No. 2004/0203145 A1, incorporated herein by reference.

The siRNA comprises a pharmacologically effective amount of a siRNA. A pharmacologically or therapeutically effective amount refers to that amount of a siRNA effective to produce the intended pharmacological, therapeutic or preventive result. The phrases “pharmacologically effective amount” and “therapeutically effective amount” or simply “effective amount” refer to that amount of a RNA effective to produce the intended pharmacological, therapeutic or preventive result. For example, if a given clinical treatment is considered effective when there is at least a 20% reduction in a measurable parameter associated with a disease or disorder, a therapeutically effective amount of a drug for the treatment of that disease or disorder is the amount necessary to effect at least a 20% reduction in that parameter.

Suitable amounts of siRNA must be introduced and these amounts can be empirically determined using standard methods. Typically, effective concentrations of individual siRNA species in the environment of a cell will be about 50 nanomolar or less 10 nanomolar or less, or compositions in which concentrations of about 1 nanomolar or less can be used. In other embodiment, methods utilize a concentration of about 200 picomolar or less and even a concentration of about 50 picomolar or less can be used in many circumstances.

In general a suitable dosage unit of siRNA will be in the range of 0.001 to 0.25 milligrams per kilogram body weight of the recipient per day, or in the range of 0.01 to 20 micrograms per kilogram body weight per day, or in the range of 0.01 to 10 micrograms per kilogram body weight per day, or in the range of 0.10 to 5 micrograms per kilogram body weight per day, or in the range of 0.1 to 2.5 micrograms per kilogram body weight per day.

The siRNA can be administered once daily. However, the siRNA formulation may also be dosed in dosage units containing two, three, four, five, six or more sub-doses administered at appropriate intervals throughout the day. In that case, the siRNA contained in each sub-dose must be correspondingly smaller in order to achieve the total daily dosage unit. The dosage unit can also be compounded for a single dose over several days, e.g., using a conventional sustained release formulation which provides sustained and consistent release of the siRNA over a several day period. Sustained release formulations are well known in the art. In this embodiment, the dosage unit contains a corresponding multiple of the daily dose. Regardless of the formulation, the pharmaceutical composition must contain siRNA in a quantity sufficient to inhibit expression of the target gene in the animal. The composition can be compounded in such a way that the sum of the multiple units of siRNA together contain a sufficient dose.

Data can be obtained from cell culture assays to formulate a suitable dosage range. The dosage of compositions of the invention lies within a range of circulating concentrations that include the ED₅₀ (as determined by known methods) with little or no toxicity. The dosage may vary within this range depending upon the dosage form employed and the route of administration utilized. For any compound used in the method of the invention, the therapeutically effective dose can be estimated initially from cell culture assays. Levels of dsRNA in plasma may be measured by standard methods, for example, by high performance liquid chromatography.

The method can be carried out by addition of the siRNA compositions to any extracellular matrix in which cells can live provided that the siRNA composition is formulated so that a sufficient amount of the siRNA can enter the cell to exert its effect. For example, the method is amenable for use with cells present in a liquid such as a liquid culture or cell growth media, in tissue explants, or in whole organisms, including animals, such as mammals and especially humans.

### Delivery Methods

DNA sequences encoding an antisense strand of a siRNA specific for a target sequence of a gene are introduced into mammalian cells for expression. To target more than one sequence in the gene (such as different promoter region sequences and/or coding region sequences), separate siRNA-encoding DNA sequences specific to each targeted gene sequence can be introduced simultaneously into the cell. In accordance with another embodiment, mammalian cells may be exposed to multiple siRNAs that target multiple sequences in the gene.

The siRNA of this invention can be administered by any means known in the art such as by parenteral routes, including intravenous, intramuscular, intraperitoneal, subcutaneous, transdermal, airway (aerosol), rectal, vaginal and topical (including buccal and sublingual) administration. In some embodiments, the pharmaceutical compositions are administered by intravenous or intraparenteral infusion or injection.

In one embodiment, the invention features the use of methods to deliver the nucleic acid molecules of the instant invention to the central nervous system and/or peripheral nervous system. Experiments have demonstrated the efficient in vivo uptake of nucleic acids by neurons. As an example of local administration of nucleic acids to nerve cells, Sommer et al., 1998, Antisense Nuc. Acid Drug Dev., 8, 75, describe a study in which a 15mer phosphorothioate antisense nucleic acid molecule to c-fos is administered to rats via microinjection into the brain. As an example of systemic administration of nucleic acid to nerve cells, Epa et al., 2000, Antisense Nuc. Acid Drug Dev., 10, 469, describe an in vivo mouse study in which beta-cyclodextrin-adamantane-oligonucleotide conjugates were used to target the p75 neurotrophin receptor in neuronally differentiated PC12 cells. Following a two week course of IP administration, pronounced uptake of p75 neurotrophin receptor antisense was observed in dorsal root ganglion (DRG) cells. In addition, a marked and consistent down-regulation of p75 was observed in DRG neurons. Additional approaches to the targeting of nucleic acid to neurons are described in Broaddus et al., 1998, J. Neurosurg., 88(4), 734; Karle et al., 1997, Eur. J. Pharmocol., 340(2/3), 153; Bannai et al., 1998, Brain Research, 784(1,2), 304; Rajakumar et al., 1997, Synapse, 26(3), 199; Wu-pong et al., 1999, BioPharm, 12(1), 32; Bannai et al., 1998, Brain Res. Protoc., 3(1), 83; Simantov et al., 1996, Neuroscience, 74(1), 39. Nucleic acid molecules of the invention are therefore amenable to delivery to and uptake by neural cells.

The delivery of nucleic acid molecules of the invention, targeting the candidate gene is provided by a variety of different strategies. Traditional approaches to CNS delivery that can be used include, but are not limited to, intrathecal and intracerebroventricular administration, implantation of catheters and pumps, direct injection or perfusion at the site of injury or lesion, injection into the brain arterial system, or by chemical or osmotic opening of the blood-brain barrier. Other approaches can include the use of various transport and carrier systems, for example though the use of conjugates and biodegradable polymers. Furthermore, gene therapy approaches, for example as described in Kaplitt et al., U.S. Pat. No. 6,180,613 and Davidson, WO 04/013280, can be used to express nucleic acid molecules in the CNS.

The method comprises introducing the siRNA into the appropriate cell. The term “introducing” encompasses a variety of methods of introducing DNA into a cell, either in vitro or in vivo. Such methods include transformation, transduction, transfection, and infection. Vectors are useful and preferred agents for introducing DNA encoding the siRNA molecules into cells. The introducing may be accomplished using at least one vector. Possible vectors include plasmid vectors and viral vectors. Viral vectors include retroviral vectors, lentiviral vectors, or other vectors such as adenoviral vectors or adeno-associated vectors. In one embodiment, the DNA sequences are included in separate vectors, while in another embodiment, the DNA sequences are included in the same vector. The DNA sequences may be inserted into the same vector as a multiple cassettes unit. Alternate delivery of siRNA molecules or DNA encoding siRNA molecules into cells or tissues may also be used in the present invention, including liposomes, chemical solvents, electroporation, viral vectors, pinocytosis, phagocytosis and other forms of spontaneous or induced cellular uptake of exogenous material, as well as other delivery systems known in the art.

Suitable promoters include those promoters that promote expression of the interfering RNA molecules once operatively associated or linked with sequences encoding the RNA molecules. Such promoters include cellular promoters and viral promoters, as known in the art. In one embodiment, the promoter is an RNA Pol III promoter, which preferably is located immediately upstream of the DNA sequences encoding the interfering RNA molecule. Various viral promoters may be used, including, but not limited to, the viral LTR, as well as adenovirus, SV40, and CMV promoters, as known in the art.

In one embodiment, the invention uses a mammalian U6 RNA Pol III promoter, and more preferably the human U6snRNA Pol III promoter, which has been used previously for expression of short, defined ribozyme transcripts in human cells (Bertrand et al., 1997; Good et al., 1997). The U6 Pol III promoter and its simple termination sequence (four to six uridines) were found to express siRNAs in cells. Appropriately selected interfering RNA or siRNA encoding sequences can be inserted into a transcriptional cassette, providing an optimal system for testing endogenous expression and function of the RNA molecules.

### Expression Measurement

Expression of a target gene can be determined by any suitable method now known in the art or that is later developed. It can be appreciated that the method used to measure the expression of a target gene will depend upon the nature of the target gene. For example, when the target gene encodes a protein the term “expression” can refer to a protein or transcript derived from the gene. In such instances the expression of a target gene can be determined by measuring the amount of mRNA corresponding to the target gene or by measuring the amount of that protein. Protein can be measured in protein assays such as by staining or immunoblotting or, if the protein catalyzes a reaction that can be measured, by measuring reaction rates. All such methods are known in the art and can be used. Where the gene product is an RNA species expression can be measured by determining the amount of RNA corresponding to the gene product. The measurements can be made on cells, cell extracts, tissues, tissue extracts or any other suitable source material.

The determination of whether the expression of a target gene has been reduced can be by any suitable method that can reliably detect changes in gene expression. Typically, the determination is made by introducing into the environment of a cell undigested siRNA such that at least a portion of that siRNA enters the cytoplasm and then measuring the expression of the target gene. The same measurement is made on identical untreated cells and the results obtained from each measurement are compared.

### EXAMPLES

### Example 1

### Screening for siRNAs Targeting CREB and PPI Using Neuro 2A Cell

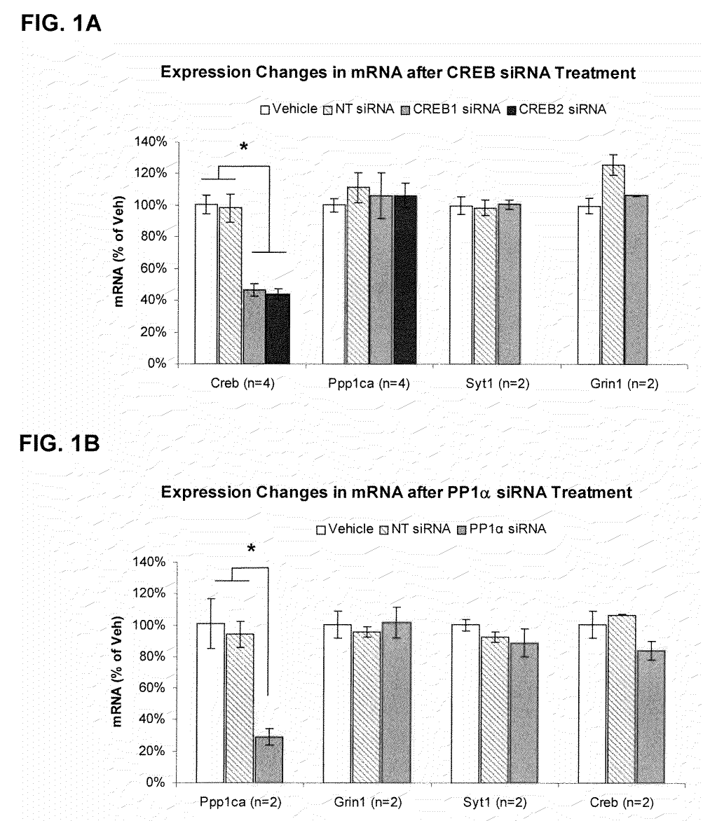

A set of siRNAs targeting CREB and the \acute{α}-isoform of PP1 were screened in the Neuro2A mouse neuroblastoma cell line. Several suitable siRNA's that could efficiently target CREB and PP1\acute{α} without affecting the mRNA levels of several control genes were identified (FIG. 1).

In vivo grade siSTABLE siRNA (Dharmacon Inc., Lafayette, USA). siRNA's were chemically modified to enhance stability. A 21mer siSTABLE non-targeting siRNA was used as control (sense strand: 5′-UAGCGACUAAACACAUCAAUU-3′; (SEQ ID NO:1) anti-sense strand: 5′-UUGAUGUGUUUAGUCGCUAUU-3′) (SEQ ID NO:2) (Dharmacon Inc., Lafayette, USA). siRNAs was designed using a multi component rational design algorithm (Reynolds, A. et al. Nat Biotechnol 22, 326-30 (2004)).

Real-Time PCR. Neuro 2A cells were treated with 100 nM siSTABLE siRNA and Dharmafect 3 carrier (Dharmacon). RNA was isolated using the QIAgen RNeasy kit (Qiagen) according to the manufacturer's specifications. cDNA was generated using TaqMan Reverse transcriptase kit (Applied Biosystems). cDNA was synthesized and real-time PCR performed using the ABI prism and SDS 2.1 software. ABI assays on demand (Applied Biosystems) were used for CREB, Synaptotagmin I (SYT1), PP1α, NR1 and TATA binding protein (TBP), respectively. qPCR reactions were run in triplicate and CT values averaged. Data was then normalized to TATA binding protein (TBP) and the ACT values determined as percentage of vehicle treated controls. Data shown is the mean+/−stdev.

A set of four non-modified siRNA's were tested against CREB and PP1α in vitro using Neuro 2a cells.

Neuro2A cells were treated with CREB siRNA or non-targeting control siRNA and mRNA levels evaluated 24 hours later. ANOVA followed by Scheffe's pair-wise comparison revealed that CREB siRNA1 and CREB siRNA2 significantly reduced mRNA levels of CREB (p<0.05 for CREB vs. both vehicle and non-targeting siRNA). In contrast, mRNA levels of Synaptotagmin I (Syt1), the NMDA receptor subunit 1 (Grin1) and protein phosphatase 1 (Ppp1ca) were not significantly affected by treatment with non-targeting or CREB siRNA (p>0.05 for all comparisons). Significant knockdown of CRE [symbol] mRNA was also observed 48 h and 72 h after siRNA treatment.

FIG. 1a shows the mRNA levels after treatment with siSTABLE CREB siRNA. The mean±stdev of two to four experimental replications are shown. Open bars: vehicle; stripped bars: non-targeting siRNA; grey bars: CREB1 siRNA; black bars: CREB2 siRNA.

Neuro2A cells were treated with PP1\acute{α} siRNA1 or non-targeting control siRNA by a similar method. FIG. 1b shows mRNA levels after treatment with siSTABLE PP1\acute{α} siRNA. The mean±sem of two replications are shown. Open bars: vehicle; stripped bars: non-targeting siRNA; grey bars: PP1\acute{α} siRNA. ANOVA followed by Scheffe's pair-wise comparison revealed that PP1\acute{α} siRNA1 significantly reduced mRNA levels of PP1\acute{α} (p<0.05 for PP1\acute{α} vs. both vehicle and non-targeting siRNA). mRNA levels of Synaptotagmin I (Syt1), the NMDA receptor subunit 1 (Grin1) and CREB (Creb) were not significantly affected by treatment with PP1\acute{α} siRNA (p>0.05 for all comparisons). Significant knockdown of PP1 [symbol] mRNA was also observed 48 h and 72 h after siRNA treatment.

bDNA assay. mRNA levels of CREB1 and PP1α were quantified using the QuantiGene bDNA assay kit (Bayer) according to the manufacturer's specifications. mRNA levels were normalized to a vehicle treated control group. Three experimental replications were run and the mean±sem of knockdown efficiency determined for each siRNA.

Several siRNA's showed similar efficacy in reducing CREB and PP1α mRNA levels (≧60%), and the following siRNA's were chosen for further in vivo characterization:

| | |
|---|---|
| CREB siRNA1 sense strand | |
| 5′-CAAUACAGCUGGCUAACAAUU-3′; | SEQ ID NO:3 |

CREB siRNA1 anti-sense strand

| | |
|---|---|
| 5′-UUGUUAGCCAGCUGUAUUGUU-3′; | SEQ ID NO:4 |

CREB siRNA2 sense strand sense strand

| | |
|---|---|
| 5′-GCAAGAGAAUGUCGUAGAAUU-3′; | SEQ ID NO:5 |
| CREB siRNA2 anti-sense strand | |
| 5′-UUCUACGACAUUCUCUUGCUU-3′; | SEQ ID NO:6 |
| PP1α sense strand | |
| 5′-UAGCGACUAAACACAUCAAUU-3′; | SEQ ID NO:7 |
| PP1α anti-sense strand | |
| 5′-UUGAUGUGUUUAGUCGCUAUU-3′; | SEQ ID NO:8 |

### Example 2

### In Vivo Delivery of Synthetic CREB siRNA in Mice

In vivo delivery of synthetic siRNA in the CNS is hampered by limited diffusion and uptake.

Subjects. Young-adult (10-12 weeks old) C57BL/6 male mice were used. Upon arrival, mice were group-housed (5 mice) in standard laboratory cages and maintained on a 12:12 hours light-dark cycle. The experiments were always conducted during the light phase of the cycle. After surgery for hippocampal cannulation, mice were single housed in individual cages and maintained so till the end of the experiment. With the exception of training and testing times, the mice had ad lib access to food and water. Mice were maintained and bred under standard conditions, consistent with National Institutes of Health (NIH) guidelines and approved by the Institutional Animal Care and Use Committee.

Animal surgery and siRNA injection. For the injection of siRNA, mice were anesthetized with 20 mg/kg Avertin and implanted with a 33-gauge guide cannula bilateraly into the dorsal hippocampus {coordinates: A=−1.8 mm, L=+/−1.5 mm to a depth of 1.2 mm; (Franklin, K. & Paxinos, G. The Mouse Brain in Stereotaxic Coordinates. (1997). Five to seven days after recovery from surgery, animals were injected with siRNA. siRNA was diluted to 0.5 μg per μl in 5% glucose and mixed with 6 equivalents of a 22 kDa linear polyethyleneimine (PEI) (Fermentas).

A linear 22 kDa PEI was used to facilitate in vivo RNAi because it has good transfection efficiency, if used for gene-transfer of plasmid DNA in the CNS, and no CNS toxicity (Tan, P. H., et al., Gene Ther 12, 59-66 (2005); Ouatas, T., et al, Int J Dev Biol 42, 1159-64 (1998); Goula, D. et al. Gene Ther 5, 712-7 (1998)).

After 10 min of incubation at room temperature, 2 μl of the siRNA mixture was injected into each hippocampus through an infusion cannula that was connected to a micro-syringe by a polyethylene tube. The entire infusion procedure took ˜2 min, and animals were handled gently to minimize stress. A total of 3 infusions of siRNA were given over a period of 3 days (1 μg si NA per hippocampus per day). Mice were trained 3 days after the last siRNA injection and tested 24 hours later. Similarly, protein levels of CREB and PP1\acute{α} were tested 3 days after the last siRNA treatment.

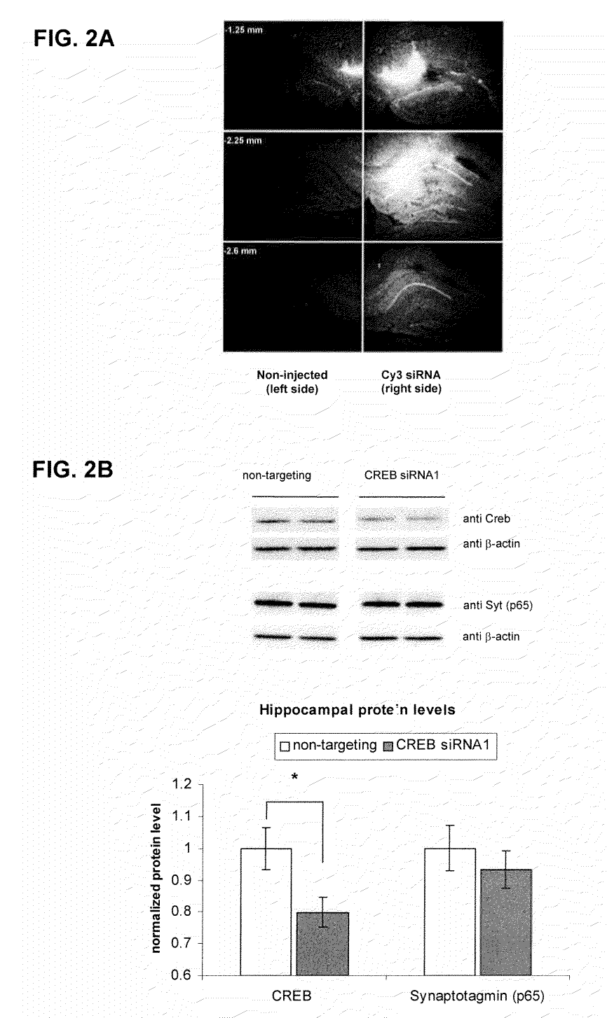

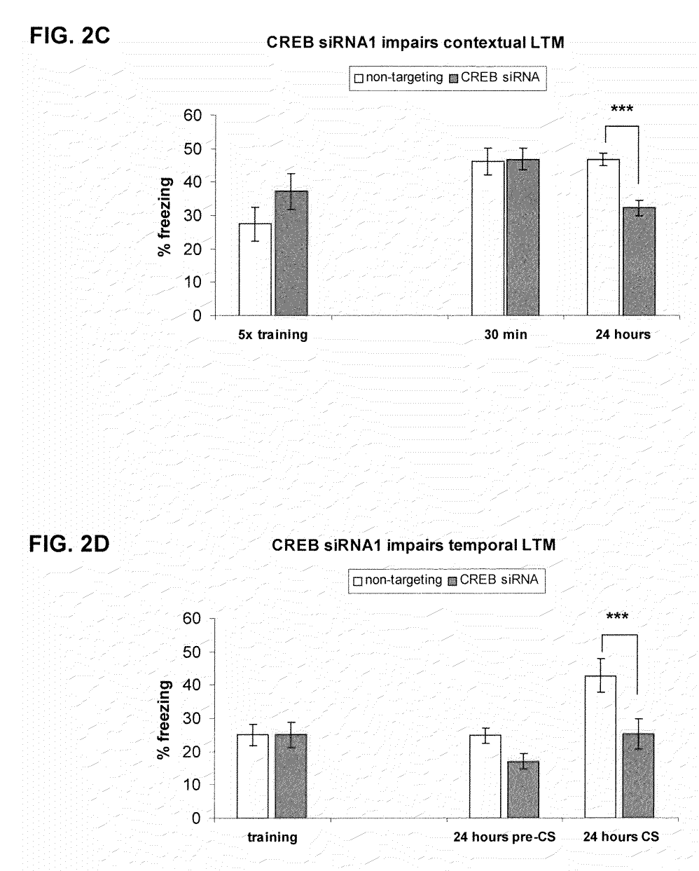

Mice were injected with Cy3 labeled siRNA and carrier, and fluorescence monitored 24 h later (FIG. 2a). For the injection of Cy3 labeled siRNA, mice were anesthetized with 20 mg/kg Avertin and 0.5 μg siRNA polyethyleneimine mix were injected at 6 sites to cover most of the hippocampal formation. Animals were sacrificed 24 h after siRNA injection. Frozen brains were sliced into 15 μm sections and images of Cy3 fluorescence acquired using a Zeiss Axioplan 2 microscope.

FIG. 2a is a picture of the coronal sections of hippocampus injected with Cy3 labeled siRNA and 22 kDa polyethyleneimine carrier. Cy3 labeling was visible several mm distal to the injection sites and was concentrated to the pyramidal cell layer. Cy3 labeling was visible throughout the dorsal hippocampus and was considerably spread from the injection sites. Importantly, Cy3 labeling was visible in the pyramidal cell layer of CA1 neurons, indicating uptake of siRNA into neurons. Note labeling of neurons at the contra-lateral, non-injected site, as well as in the ventral part of the hippocampus, indicating uptake of siRNA. Thus, the synthetic 21mer siRNA was targeted efficiently to hippocampal neurons in vivo.

Histology. CREB and non-targeting siRNA injected animals were sacrificed one day after the behavioral experiments. Frozen brains were sliced into 15 μm sections and stained with Cresyl violet. Hippocampal morphology was evaluated on photographs of serial sections.

Western-Blot Analysis. Mice were sacrificed by cervical dislocation, the hippocampi quickly removed and frozen on dry ice. Each hippocampus was lysed in 300 μl RIPA protein lysis buffer (Upstate Biotechnology) containing Roche complete protease inhibitor tablet. Protein concentrations were determined using Biorad DC compatible protein assay kit (Biorad) according to the manufacturer's instructions. 20 or 40 μg lysate were separated by SDS-PAGE and blotted onto Nitrocellulose membranes. Immunodetection of proteins was performed according to standard procedures using polyclonal antibodies against CREB and PP1α (Upstate Biotechnology 06-863 and 07-273, respectively) and Synaptotagmin I (p65) (Sigma S2177). Blots were stripped and normalized against β-actin (Sigma A2066).

Western blot analysis using an antibody against an n-terminal epitope of CREB (amino acids 5-24) revealed that siRNA1 significantly reduced hippocampal CREB protein levels without affecting Synaptotagmin I expression at this time-point (CREB: p<0.05, F_{1,11}=6.28; Synaptotagmin I: p=0.49, F_{1,11}=0.51 FIG. 2b). FIG. 2b shows a bar graph of hippocampal protein levels of CREB and Synaptotagmin after siRNA injection. CREB siRNA treated mice had significantly reduced levels of hippocamal CREB, whereas siRNA did not affect protein levels of Synaptotagmin (CREB: P<0.05, F(1,11)=6.279; Synaptotagmin: p=0.49, F(1,11)=0.51; n=6 for both groups).

### Example 3

### Effect of siRNA Mediated Knockdown of CREB on Contextual and Trace Conditioning

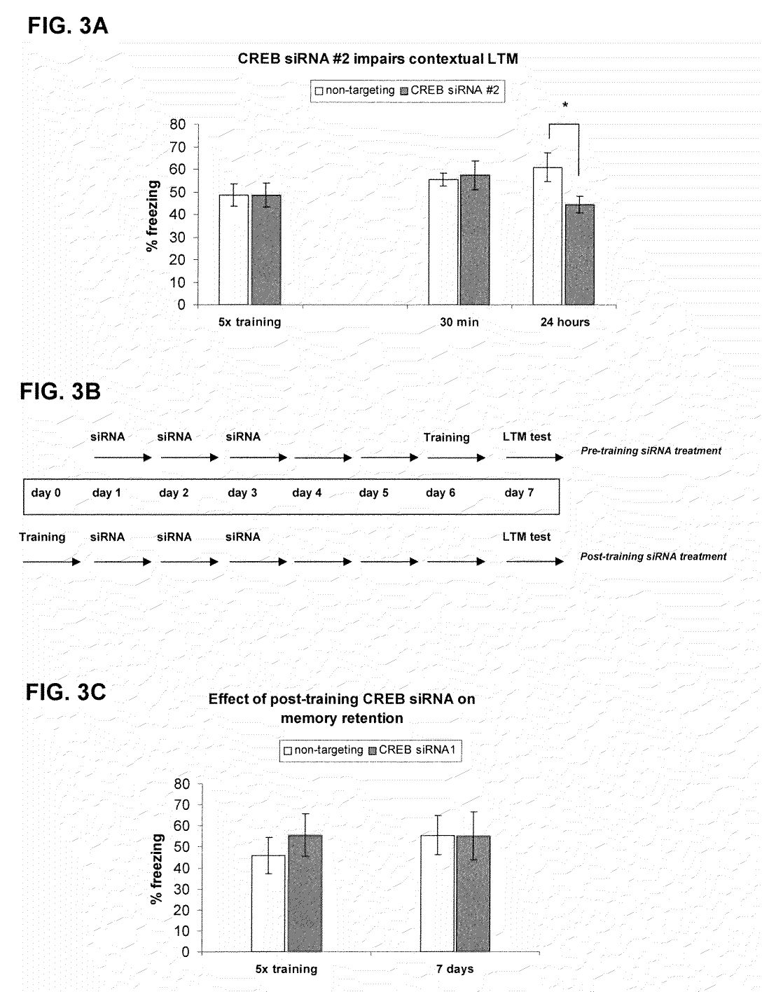

The effect of siRNA mediated knockdown of CREB on contextual fear conditioning was tested. siRNA targeting a region common to all splice variants of the CREB gene (1114-1132 of NM_{—}009952, corresponding to exon 7 of the CREB gene) was used. Nomenclature according to (Lonze and Ginty, 2002, Neuron, 35:605-623)). Mice were treated with CREB siRNA1 or a non-targeting control siRNA once daily for 3 consecutive days. Behavioral testing was initiated 3 days later (see also FIG. 3b). This design was chosen based on pilot experiments on siRNA knockdown in hippocampus, and because previous studies have indicated that gene-knockdown by siRNA duplexes takes several days to develop in CNS ((Salahpour et al., 2007, Biol. Psychiatry 61:65-69) Tan et al., 2005, Gene Therapy 12:59-66; Thakker et al., 2004, Proc. Natl. Acad. Sci USA 101:17270-17275).

Contextual conditioning was essentially done as described (Bourtchuladze, R. et al. Cell 79, 59-68 (1994); Bourtchouladze et al, Learn Mem 5, 365-374 (1998)). Mice were placed in the conditioning chamber (Med Associates, Inc., VA) and allowed to explore for 2 min. Then a total of two (weak memory) or five (strong memory) foot-shocks were delivered (0.5 mA, 2 s duration) with an inter-trial interval of 1 min. Freezing was scored for 30 s after the last foot-shock (immediate freezing). The mice were then returned to their home-cage. Memory was tested after 30 min (STM) or 24 h (LTM). To assess contextual memory, freezing behavior was scored for 3 min in intervals of 5 s in the chamber in which the mice were trained.

Statistical Analysis. All behavioral experiments were designed and performed in a balanced fashion, meaning that (i) for each experimental condition we used an equal number of experimental and control mice; (ii) each experimental condition was replicated several times, and replicate days were added to generate final number of subjects. The proceeding of each session was filmed. In each experiment, the experimenter was unaware (blind) to the treatment of the subjects during training and testing. Data were analyzed by Student's unpaired t test using a software package (StatView 5.0.1; SAS Institute, Inc). Trace conditioning was analyzed by repeated measures ANOVA followed by contrast analysis using Jmp software. All values in the text and figures are expressed as mean±sem.

Mice were treated with non-targeting or CREB siRNA1 and trained with 5 CS-US pairings to induce robust contextual memory. When tested in the training context, CREB siRNA1-injected mice demonstrated significantly reduced long-term memory (LTM) tested 24 h after training (p<0.001, n=17 for both groups) (FIG. 2c). In contrast, CREB siRNA1 did not affect short-term contextual memory (STM) 30 min after training or immediate memory during the training procedure (STM: p=0.89, n=8 for both groups; immediate freezing: p=0.2, n=17 for both groups; FIG. 2c). Importantly, contextual memory in non-targeting control siRNA-treated animals was similar to that observed in non-treated mice (53.9±5.2%, n=20).

CREB and non-targeting siRNA in combination with linear PEI did not cause any obvious damage to the hippocampal formation. This was underscored by our behavioral results.

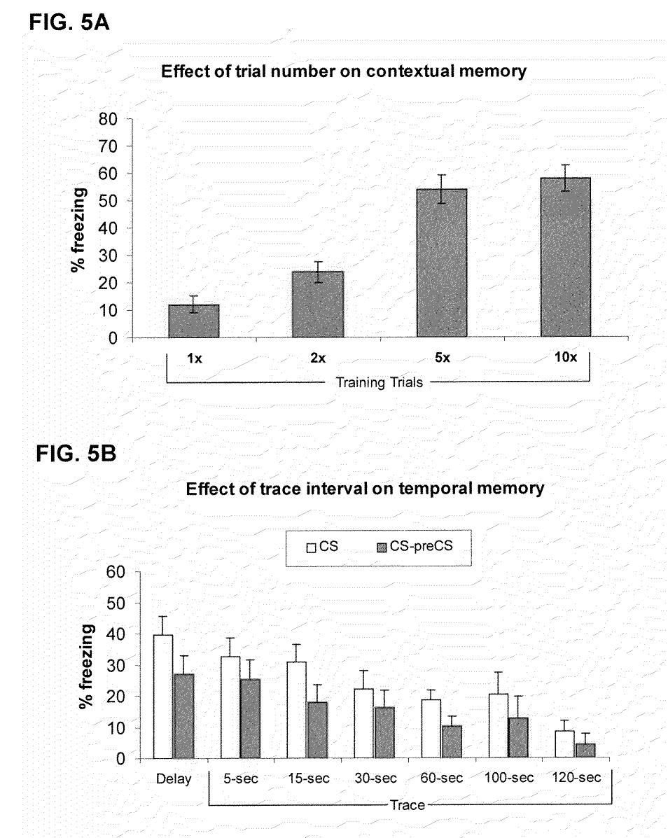

Both contextual and temporal memory requires the hippocampus, but little is known about the molecular mechanisms underlying temporal memory formation. To test if contextual and trace fear memory share a requirement for CREB in the hippocampus, we studied the effects of CREB siRNA1 in trace conditioning (FIG. 2d). CREB and non-targeting siRNA injected mice were trained with a trace interval of 15 seconds and memory for the tone CS tested 24 hours later). (FIG. 5)

For trace conditioning, the mouse was placed in the conditioning chamber for 2 min before the onset of the conditioned stimulus (CS), a tone, which lasted for 20 s at 2800 Hz, 75 dB. After a 15 s interval the shock unconditioned stimulus (US) was presented. In total, 3 CS-US pairings were presented with a 1 min interval between trials to induce a strong trace memory. Facilitation of temporal memory was assessed using a single CS-US pairing with a 60 sec trace interval. After an additional 30 s in the chamber, the mouse was returned to its home cage. Mice were tested at 24 hours after training. Testing was done in a novel chamber (a modified home-cage). Memory for trace conditioning was assessed by scoring freezing behavior which was defined as the complete lack of movement in intervals of 5 s. Freezing was scored for 2 min before tone CS onset (preCS) and for 20 s during tone presentation (CS).

Repeated measures ANOVA with tone CS presentation as within factor revealed a significant treatment by trial interaction (F_{3,128}=8.39, p<0.0001). CREB siRNA1 infused mice demonstrated significantly impaired memory for the tone CS (preCS: p=0.15, CS: p<0.005, n=34 for non-targeting and n=32 for CREB siRNA1 treated mice). Importantly, non-targeting but not CREB siRNA1 treated mice had formed a memory for the tone CS (Effect of tone CS presentation: p<0.001 for non-targeting and p=0.14 for CREB siRNA1, respectively). As for contextual memory, trace memory in control siRNA treated mice was similar to non-treated animals (preCS: 19.2±7.0%, CS: 40.0±6.5%, n=10). Thus, CREB is required not only for contextual, but also for temporal LTM.

Synthetic siRNA may produce significant off-target activity. Such off-target effects are siRNA sequence-specific and target independent (Jackson, A. L. et al. Nat Biotechnol 21, 635-7 (2003)). Although our results show that CREB siRNA specifically interfered with LTM but not STM, long-term memory could have been affected by non-specific targeting as well.

To address this, we performed two experiments: (i) tested a second siRNA against CREB and (ii) injected siRNA after training.

To confirm the specificity of the results, a second siRNA against CREB targeting a different region of the CREB gene was injected (1114-1132 of NM_{—}009952, corresponding to exon 9 of the CREB gene). CREB siRNA2 did not show any obvious off-target activity when tested in Neuro2A cells (FIG. 1a). Similar to CREB siRNA1, CREB siRNA2 impaired contextual LTM, but not STM or learning (LTM: p<0.05, n=12 for both groups, STM: p=0.794, n=6 for both groups, immediate freezing: p=0.99, n=12 for both groups; FIG. 3a). In parallel biochemical experiments, infusion of siRNA2 significantly reduced levels of hippocampal CREB at the time of training (1.00±0.07 vs. 0.73±0.05, p<0.05, F_{1,11}=9.65; n=6 for non-targeting and CREB siRNA2 treated mice, respectively).

Previous results on the role of CREB in the dorsal hippocampus for memory formation have indicated that CREB around the time of training, but not at delays of more than 1 day after training, is required for spatial memory formation. Thus, to test the temporal specifics of the effect of CREB siRNA on contextual memory formation, cannulated mice were trained in 5US context conditioning and started siRNA infusion 24 hours later. Similar as in all other experiments, mice were repeatedly treated with siRNA over 3 days and memory tested 4 days after the last siRNA injection (FIG. 3b). Post-training infusions of CREB or non-targeting control siRNA did not affect contextual LTM (LTM (7 day memory: p=0.99, immediate freezing: p=0.48, n=8 for both groups FIG. 3c). Thus, siRNA knockdown of CREB during conditioning specifically impaired long-term memory, while reduction of CREB after training does not affect memory retention. Contextual memory is highly sensitive to post-training lesions of the dorsal hippocampus within a period of two weeks after behavioral training (Anagnostaras et al., 2001, Hippocampus 11:8-17). Consequently, if siRNA causes damage to hippocampus, it would be expected to impair contextual memory when injected after the training experience. Our results therefore also show that the siRNAs tested here are unlikely to cause significant non-specific damage to hippocampal neurons in vivo, as has been suggested for a subset of shRNAs expressed from viral vectors (Alvarez et al., 2006, J. Neurosci 26:7820-7825).

The results show that CREB is required for hippocampal memory formation. siRNA mediated knockdown of CREB in hippocampus impaired LTM for contextual and auditory trace fear conditioning, while leaving STM intact. Contextual and temporal memory both share the requirement for CREB in the hippocampus.

In parallel biochemical experiments, infusion of siRNA2 significantly reduced levels of hippocampal CREB (1.00±0.07 vs. 0.73±0.05, p<0.05, F_{1,11}=9.65; n=6 for non-targeting and CREB siRNA2 treated mice, respectively one-way ANOVA.

### Example 4

### Effect of siRNA Mediated Knockdown of PP1 on Contextual and Trace Conditioning

To further evaluate the suitability of the siRNA approach to the study of hippocampal memory formation the memory suppressor gene protein phosphatase 1 (PP1) was targeted. PP1 acts as a negative regulator of CaMKII\acute{α} and the AMPA ionotrophic glutamate receptors (reviewed in (Lisman and Zhabotinsky, 2001, Neuron 31:191-201)). PP1 dephosphorylates CREB activated by PKA or CaMKIV and inhibits CREB activation during memory formation (Bito et al., 1996, Cell 87:1203-1214)(Lonze, B. E. & Ginty, D. D. 2002, Neuron 35, 605-23; Genoux, D. et al. 2002, Nature 418:970-5). Previous results have indicated that genetic inhibition of PP1 by over-expression of inhibitor-1 in forebrain facilitates object recognition memory and enhances CRE-dependent transcription during memory formation (Genoux, D. et al. 2002, Nature 418:970-5). Thus, because siRNA knockdown of CREB inhibited memory formation, siRNA-mediated knockdown of PP1 should facilitate contextual and temporal memory. At least three isoforms of PP1 are expressed in rodent hippocampus (\acute{α}, β, and γ1; (da Cruz e Silva et al., 1995, J. Neurosci. 15:3375-3389)). The \acute{α}-subunit of PP1 (PP1\acute{α}) was targeted because of its dendritic as well as nuclear localization and abundance in the hippocampal formation (Ouimet et al., 1995, Proc. Natl, Acad Sci USA 92:3396-3400).

Mice were trained with a contextual conditioning paradigm that induces weak memory (FIG. 5a, also see Tully, T., et al., Nat Rev Drug Discov 2, 267-77 (2003)). FIG. 5a shows the effect of number of trials on contextual memory formation. Mice were trained with increasing numbers of CS-US pairings and contextual memory assessed 4 days later. Training with 1× or 2×CS-US pairings induced sub-maximal memory (n=22 for 1×, n=20 for 2×, n=20 for 5×, n=22 for 10× shock US presentations, respectively).

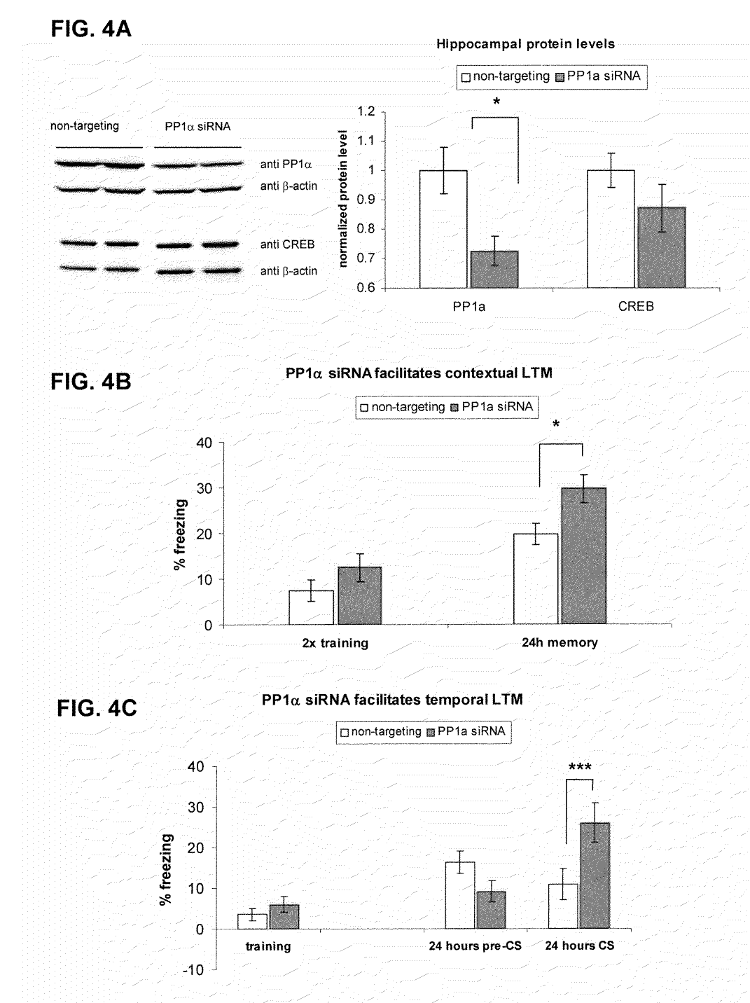

Mice were treated with PP1\acute{α} or control siRNA in an identical way as described for CREB siRNA and then trained in contextual fear conditioning with 2 CS-US pairings to induce weak contextual memory (FIG. 5a, (Tully et al., 2003, Nat. Rev. Drug Discov. 2:267-277)). PP1\acute{α} siRNA injected animals demonstrated significantly enhanced freezing at 24 h after training (LTM: p<0.05, n=29 for non-targeting and n=32 for PP1 [symbol] siRNA treated mice, FIG. 4b). Importantly, PP1\acute{α} siRNA had no effect on immediate freezing during the training procedure (immediate freezing: p=0.20, FIG. 4b). Thus, infusion of PP1\acute{α} siRNA into hippocampus facilitated contextual LTM.

Consistent with these findings, PP1\acute{α} protein levels were reduced in the hippocampus as a result of PP1\acute{α} siRNA injections, while protein levels of CREB were not affected (PP1\acute{α}: F_{1,11}=8.72, p<0.05; CREB: F_{1,11}=1.74, p=0.22; n=6 for non-targeting siRNA, n=6 and n=5 for PP1 [symbol] siRNA and CREB protein levels respectively FIG. 4a). PP1\acute{α} siRNA did not cause any obvious alteration in hippocampal morphology.

A role for PP1 in trace conditioning was also investigated. Trace conditioning becomes increasingly difficult as the time interval between CS and US increases. In fact, C57BL/6 mice show poor memory if the trace interval between CS and US is 60 seconds or longer (FIG. 5). FIG. 5b shows the effect of the trace interval on temporal memory formation. Mice were trained in trace fear conditioning using increasingly long trace intervals and tone memory compared to delay conditioning. Trace intervals of 30 sec or longer resulted in poor long-term memory for the tone CS (n=29, n=20, n=25, n=18, n=28, n=16 and n=12 for delay conditioning and trace intervals of 5 sec, 15 sec, 30 sec, 60 sec, 100 sec, and 120 sec, respectively).

When mice were trained with one CS/US pairing and a 60 seconds trace interval, PP1\acute{α} siRNA improved trace memory (FIG. 4c). Repeated measures ANOVA revealed a significant treatment by trial interaction (F_{3,95}=4.38, p<0.01). PP1\acute{α} siRNA treated mice froze significantly more on tone (CS) than control siRNA injected mice (preCS: p=0.17, CS: p<0.005, n=23 for non-targeting control and n=25 for PP1 [symbol] siRNA treated mice). Importantly, PP1\acute{α} but not control siRNA treated mice increased their freezing response upon tone presentation (Effect of tone CS presentation: p=0.31 and p<0.005 for non-targeting and PP1\acute{α} siRNA, respectively). Thus, similarly to contextual conditioning, siRNA-mediated knockdown of hippocampal PP1\acute{α} facilitated trace conditioning.

In summary, this shows that PP1 inhibits hippocampal memory formation. siRNA mediated knockdown of PP1\acute{α} in hippocampus is sufficient to enhance both contextual and temporal memory formation. Because this facilitation of memory formation can not be explained by detrimental effects of siRNA, these findings show that the siRNA approach is amenable to the study of molecular mechanisms of memory.

### Example 5

### Screening for siRNAs Targeting Gpr12 Using Neuro 2A Cell

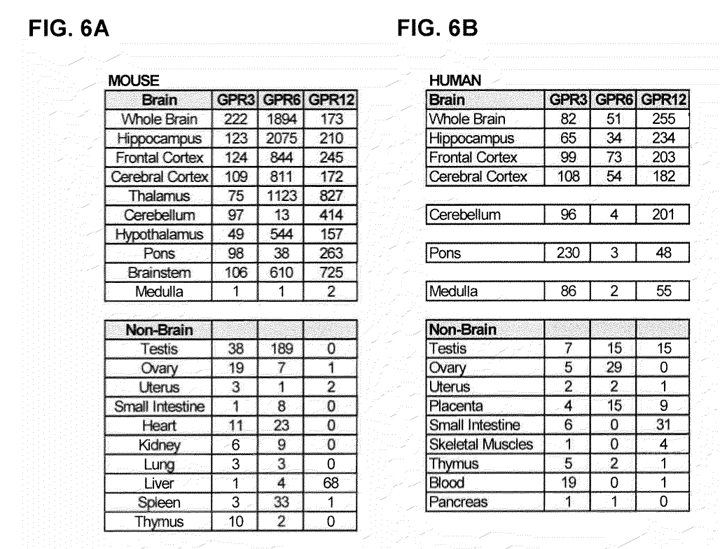

Expression profiling by real-time PCR revealed Gpr12 mRNA expression within mouse and human CNS with little expression in peripheral tissues (FIG. 6).

The sequences of the mouse Gpr12 and human Gpr12 mRNA and protein as provided in Table 1.

TABLE 1
GRP12 genes and proteins

| | | SEQ | |
|---|---|---|---|
| | | ID | |
| Name | Accession | NO: | Sequence |
| Mouse | NP_001010941 | 9 | mnedpkvnls glprdcidag apenisaavp sqgsvaesep elvvnpwdiv lcssgtlicc |
| GPR12 | | | enavvvliif hspslrapmf lligslalad llaglgliin fvfayllqse atklvtigli |
| protein | | | vasfsasvcs llaitvdryl slyyaltyhs ertvtftyvm lvmlwgtsic lgllpvmgwn |
| | | | clrdestcsv vrpltknnaa ilsisflfmf almlqlyiqi ckivmrhahq ialqhhflat |
| | | | shyvttrkgv stlalilgtf aacwmpftly sliadytyps iytyatllpa tynsiinpvi |
| | | | yafrnqeigk alcliccgci psslsgrars psdv |
| Mouse | NM_001010941 | 10 | aagggaacaa taatttgcag accggccaac tgcaatctaa gagagggagt cgcttgctgt |
| Variant 1 | | | tgtaagtctc ctccgccagc cctaacctgc ttaccccgca ttcctcctgt tcatcccgaa |
| mRNA | | | aacccggccg tttacaattc tttaggggaa agcataagaa gccgagcccc agggtcaagg |
| | | | gcgcctcggg gaagccacag gatcaaagta ggtcgccaga ctctccggcc gttcgagtgg |
| | | | gtcttcgcat gactgttgca ggcgggcgtc cacggtggcg ggctcccgcc cctcacgcag |
| | | | ctgcgacctg cgggggcgcg cgcagcctcg tggggttccc gcggatgcgc gcccggcggg |
| | | | gagcgcggag ggcggagagc cgggcgcgag caccgcagct cacctgccgc gggcgccacc |
| | | | acggacgtgc cacgcgggtg gcccgagcta ttcggcagca ctgaaggagc cacccctcgg |
| | | | ccagggcgtg ccaaggacag gggttaaaat gaacgaagac ccgaaggtca atttaagcgg |
| | | | gctgcctcgg gactgtatag atgccggtgc tccagagaac atctcagccg ctgtcccctc |
| | | | ccagggctct gttgcggagt cagaacccga gctcgttgtc aacccctggg acattgtctt |
| | | | gtgcagctca ggaaccctca tctgctgtga aaatgccgtt gtggtcctta tcatcttcca |
| | | | cagccccagc ctgcgagccc ccatgttcct actgataggc agcctggctc ttgcagacct |
| | | | gctggctggc ctgggactca tcatcaattt tgtttttgcg tacctgcttc agtcagaagc |
| | | | caccaagctg gtcaccatcg gactcattgt cgcctctttc tctgcctctg tctgcagttt |
| | | | gctggctatt actgtggacc gctacctctc gctatattac gccctgacgt accactccga |
| | | | gaggaccgtc acctttacct atgtcatgct agtgatgctc tggggaacct ccatctgcct |
| | | | ggggctgctg cccgtcatgg gctggaactg cttgagggac gagtccacct gcagcgtggt |
| | | | cagacctctc actaagaaca acgctgccat cctctccatc tccttcctct tcatgtttgc |
| | | | tctgatgctt cagctctaca tccagatttg taagattgtg atgaggcacg cccatcagat |
| | | | agccctgcag caccacttcc tggctacatc gcactatgtg actacccgga aaggggtctc |
| | | | gaccctggct ctcatcctag ggacctttgc tgcctgctgg atgcctttca ccctctattc |
| | | | cttgatcgcc gattacacct acccttcgat ctatacctat gccaccctcc tgcccgccac |
| | | | ctacaattcc atcatcaacc ctgtcattta cgctttcaga aaccaagaga tccagaaagc |
| | | | cctctgcctc atttgctgtg ggtgcatccc ttcctcgctg tctcagagag ctcggtctcc |
| | | | cagcgatgtg tagcagcctt ctcctcatag gacgctgcct ctaccaagcg ctcccacctc |
| | | | ccagggcggc cagtgatttc cttccttaaa ttctttgcac tggatctcac aagcagaagc |
| | | | aatgacatct tttagacacg tattgacagt ggaaatcatc ttaccagtgt tttttaaaaa |
| | | | aaaaacaaaa caaaactcga cttctcggct cagcattctg ttgtttggtt tgggagttag |
| | | | gatttgtttg tttgtttgct tgtttgtttg tttggagggt gtaatgggac ctcatgtggc |
| | | | catgaaatta tacaaaagtc tcgggatttt ttaacctagg cttgaaaata aatcaaagtt |
| | | | ttaaaggaaa ctggagaagg aaatactttt tctgaaggaa atactttttt ttttttaatc |
| | | | aaggtagatc ttccattctg tatgtatcta acaggatagg agctttgcca tataaccaaa |
| | | | atagtttata taattacatt tggaagggct tgtgtttatt tctaggaatt cagtaataag |
| | | | tgaccagtaa cagaggcgcg aactcctttc tttcctttca gcagtagtga ctgctcttaa |
| | | | gaatcacttt gcagtttctc tgtgttacag tttggtatgc atggttacct gtggtagtca |
| | | | gatcactaat tgcaatattg ccatgttaaa cccagaatta aaagagtcat tttttcttca |
| | | | atacagtttt tgaaatatcc tttccaaagt gagtcatgaa aaaaatgttt ccaattacat |
| | | | atgagatagc actggttaga tttgtcattg tgatttttaa aactctagac tggtggtttt |
| | | | cagaaaacaa aagagaaaat attaacagca tctattgaaa gaagatttta tttattttta |
| | | | atatattctg agagaataaa tggtgtgata ctattaagaa atatacaaac atgacttttc |
| | | | aaatctctaa aaaaaaaaaa aaaaa |
| Mouse | NM_008151 | 11 | cggcatggga gatgcaatta gccaatgtcg gttttcagcg ttttggcaag tgtgcgagtg |
| variant 2 | | | tgcatgtgcc gcctcgggag tcctgatccg tgtttccctc agagacaaac agcatttcgg |
| mRNA | | | ttgcagactt tagcttttgt ttttaattcc tgaagctcgt ggcattttga cactgatagc |
| | | | tgagcccagg gttgtctgtc tttctctgtg tgttttgcat gatcttggat tggcacccta |
| | | | ctgtacccaa acattaaaaa gcctgtcttt ccgttgaaga ggacaggggt taaaatgaac |
| | | | gaagacccga aggtcaattt aagcgggctg cctcgggact gtatagatgc cggtgctcca |
| | | | gagaacatct cagccgctgt cccctcccag ggctctgttg cggagtcaga acccgagctc |
| | | | gttgtcaacc cctgggacat tgtcttgtgc agctcaggaa ccctcatctg ctgtgaaaat |
| | | | gccgttgtgg tccttatcat cttccacagc cccagcctgc gagcccccat gttcctactg |
| | | | ataggcagcc tggctcttgc agacctgctg gctggcctgg gactcatcat caattttgtt |
| | | | tttgcgtacc tgcttcagtc agaagccacc aagctggtca ccatcggact cattgtcgcc |
| | | | tctttctctg cctctgtctg cagtttgctg gctattactg tggaccgcta cctctcgcta |
| | | | tattacgccc tgacgtacca ctccgagagg accgtcacct ttacctatgt catgctagtg |
| | | | atgctctggg gaacctccat ctgcctgggg ctgctgcccg tcatgggctg gaactgcttg |
| | | | agggacgagt ccacctgcag cgtggtcaga cctctcacta agaacaacgc tgccatcctc |
| | | | tccatctcct tcctcttcat gtttgctctg atgcttcagc tctacatcca gatttgtaag |
| | | | attgtgatga ggcacgccca tcagatagcc ctgcagcacc acttcctggc tacatcgcac |
| | | | tatgtgacta cccggaaagg ggtctcgacc ctggctctca tcctagggac ctttgctgcc |
| | | | tgctggatgc ctttcaccct ctattccttg atcgccgatt acacctaccc ttcgatctat |
| | | | acctatgcca ccctcctgcc cgccacctac aattccatca tcaaccctgt catttacgct |
| | | | ttcagaaacc aagagatcca gaaagccctc tgcctcattt gctgtgggtg catcccttcc |
| | | | tcgctgtctc agagagctcg gtctcccagc gatgtgtagc agccttctcc tcataggacg |
| | | | ctgcctctac caagcgctcc cacctcccag ggcggccagt gatttccttc cttaaattct |
| | | | ttgcactgga tctcacaagc agaagcaatg acatctttta gacacgtatt gacagtggaa |
| | | | atcatcttac cagtgttttt taaaaaaaaa acaaaacaaa actcgacttc tcggctcagc |
| | | | attctgttgt ttggtttggg agttaggatt tgtttgtttg tttgcttgtt tgtttgtttg |
| | | | gagggtgtaa tgggacctca tgtggccatg aaattataca aaagtctcgg gattttttaa |
| | | | cctaggcttg aaaataaatc aaagttttaa aggaaactgg agaaggaaat actttttctg |
| | | | aaggaaatac tttttttttt ttaatcaagg tagatcttcc attctgtatg tatctaacag |
| | | | gataggagct ttgccatata accaaaatag tttatataat tacatttgga agggcttgtg |
| | | | tttatttcta ggaattcagt aataagtgac cagtaacaga ggcgcgaact cctttctttc |
| | | | ctttcagcag tagtgactgc tcttaagaat cactttgcag tttctctgtg ttacagtttg |
| | | | gtatgcatgg ttacctgtgg tagtcagatc actaattgca atattgccat gttaaaccca |
| | | | gaattaaaag agtcattttt tcttcaatac agtttttgaa atatcctttc caaagtgagt |
| | | | catgaaaaaa atgtttccaa ttacatatga gatagcactg gttagatttg tcattgtgat |
| | | | ttttaaaact ctagactggt ggttttcaga aaacaaaaga gaaaatatta acagcatcta |
| | | | ttgaaagaag attttattta tttttaatat attctgagag aataaatggt gtgatactat |
| | | | taagaaatat acaaacatga cttttcaaat ctctaaaaaa aaaaaaaaaa a |
| Mouse | NP_032177 | 12 | mnedpkvnls glprdcidag apenisaavp sqgsvaesep elvvnpwdiv lcssgtlicc |
| GPR12 | | | enavvvliif hspslrapmf lligslalad llaglgliin fvfayllgse atklvtigli |
| protein | | | vasfsasvcs llaitvdryl slyyaltyhs ertvtftyvm lvmlwgtsic lgllpvmgwn |
| | | | clrdestcsv vrpltknnaa ilsisflfmf almlqlyiqi ckivmrhahq ialqhhflat |
| | | | shyvttrkgv stlalilgtf aacwmpftly sliadytyps iytyatllpa tynsiinpvi |
| | | | yafrnqeiqk alcliccgci psslsqrars psdv |
| Human | NM_005288 | 13 | atgaatgaag acctgaaggt caatttaagc gggctgcctc gggattattt agatgccgct |
| GPR12 | | | gctgcggaga acatctcggc tgctgtctcc tcccgggttc ctgccgtaga gccagagcct |
| mRNA | | | gagctcgtag tcaacccctg ggacattgtc ttgtgtacct cgggaaccct catctcctgt |
| | | | gaaaatgcca ttgtggtcct tatcatcttc cacaacccca gcctgcgagc acccatgttc |
| | | | ctgctaatag gcagcctggc tcttgcagac ctgctggccg gcattggact catcaccaat |
| | | | tttgtttttg cctacctgct tcagtcagaa gccaccaagc tggtcacgat cggcctcatt |
| | | | gtcgcctctt tctctgcctc tgtctgcagc ttgctggcta tcactgttga ccgctacctc |
| | | | tcactgtact acgctctgac gtaccattcg gagaggacgg tcacgtttac ctatgtcatg |
| | | | ctcgtcatgc tctgggggac ctccatctgc ctggggctgc tgcccgtcat gggctggaac |
| | | | tgcctccgag acgagtccac ctgcagcgtg gtcagaccgc tcaccaagaa caacgcggcc |
| | | | atcctctcgg tgtccttcct cttcatgttt gcgctcatgc ttcagctcta catccagatc |
| | | | tgtaagattg tgatgaggca cgcccatcag atagccctgc agcaccactt cctggccacg |
| | | | tcgcactatg tgaccacccg gaaaggggtc tccaccctgg ctatcatcct ggggacgttt |
| | | | gctgcttgct ggatgccttt caccctctat tccttgatag cggattacac ctacccctcc |
| | | | atctatacct acgccaccct cctgcccgcc acctacaatt ccatcatcaa ccctgtcata |
| | | | tatgctttca gaaaccaaga gatccagaaa gcgctctgtc tcatttgctg cggctgcatc |
| | | | ccgtccagtc tcgcccagag agcgcgctcg cccagtgatg tgtag |
| Human | NP_005279 | 14 | mnedlkvnls glprdyldaa aaenisaavs srvpavepep elvvnpwdiv lctsgtlisc |
| GPR12 | | | enaivvliif hnpslrapmf lligslalad llagiglitn fvfayllqse atklvtigli |
| protein | | | vasfsasvcs llaitvdryl slyyaltyhs ertvtftyvm lvmlwgtsic lgllpvmgwn |
| | | | clrdestcsv vrpltknnaa ilsvsflfmf almlqlyiqi ckivmrhahq ialqhhflat |
| | | | shyvttrkgv stlaiilgtf aacwmpftly sliadytyps iytyatllpa tynsiinpvi |
| | | | yafrnqeiqk alcliccgci psslaqrars psdv |

Gpr12 is widely present in the mouse CNS (FIG. 6a), with highest expression levels in thalamus, brainstem, and cerebellum, areas of the brain involved in feeding and the integration of sensory information (thalamus), motor control (cerebellum), and autonomous function (brainstem). High levels of Gpr12 were also observed in hippocampus and neocortex, two brain areas critical to memory formation (Fanselow 2005 J Comp Physiol Psychol 93, 736-744). These results are similar to those observed by in situ hybridization in mouse CNS (Ignatov 2003 J Neurosci 23, 907-914). In mouse, Gpr12 expression was below detection levels in most peripheral tissues, with the exception of the liver.

Within the human CNS Gpr12 expression was highest in hippocampus, the neocortex, and the cerebellum (FIG. 6b).

Gpr3 and Gpr6, the closest homologous of Gpr12, were present in the CNS of both mouse and human (FIG. 6a/b). However, Gpr12 mRNA levels appear to be much higher in human CNS than those of Gpr3 and 6. This is in contrast to the situation in mouse, where Gpr6 expression is very prominent in hippocampus, thalamus and neocortex.

In vivo grade siSTABLE siRNA (Dharmacon Inc., Lafayette, USA) was used for evaluation of Gpr12 function in the mouse CNS. siRNA's were chemically modified to enhance stability. A 21mer siSTABLE non-targeting siRNA was used as control.

For evaluation of siRNA efficacy, Neuro2A cells were transfected using siGENOME siRNA and Dharmafect 3 (Dharmacon, Lafayette, USA). RNA was isolated at 24 h after transfection and cDNA synthesized as described for hippocampal tissue. Per treatment, three individual RNA preparations and cDNA syntheses were performed. Target mRNA levels were determined in duplicate per cDNA replication and ΔCT values averaged for each experimental replication (n=3 RNA/cDNA preps; Each represented as the mean of two qPCR determinations).

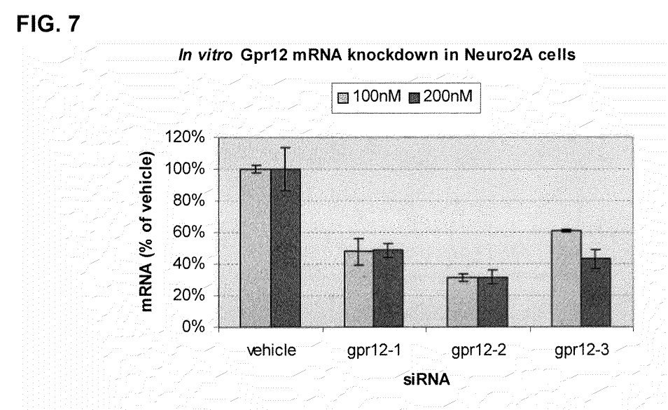

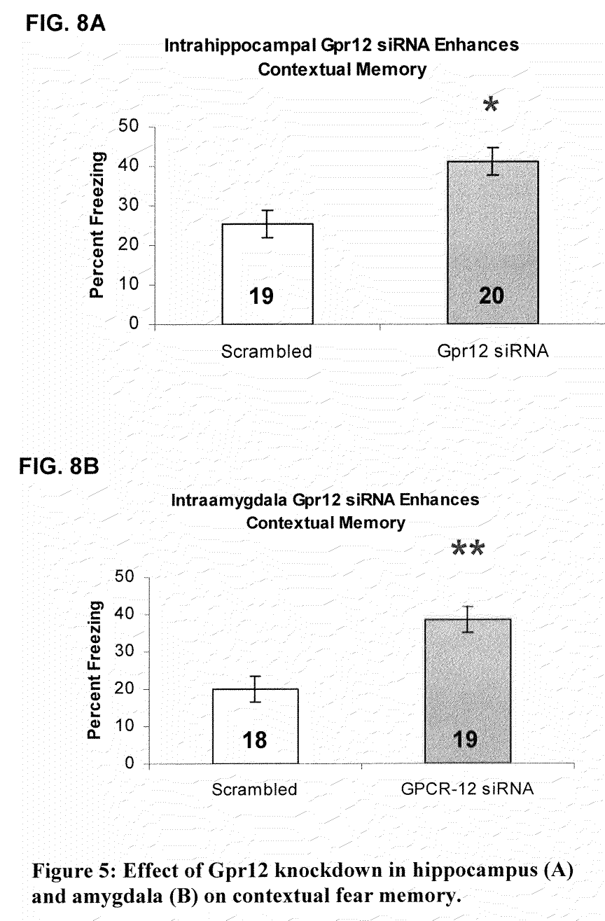

Three siRNAs were identified that efficiently reduced Gpr12 mRNA in vitro (FIG. 7). siRNA2 reduced Gpr12 mRNA levels to 31% of vehicle control at 24 h after treatment and was chosen for in vivo evaluation of Gpr12. In vivo grade siSTABLE siRNA for Gpr12-2 siRNA was obtained from Dharmacon (Lafayette, USA).

Several non-modified (siGENOME) siRNA's against Gpr12 were tested by bDNA assay (QuantiGene bDNA assay kit, Bayer) in vitro using Neuro 2a cells. siRNA was designed using a multi component rational design algorithm (Reynolds et al., (2004). Nat Biotechnol 22, 326-330) and controlled for specificity towards Gpr12 by BLAST search.

The following siRNAs were chosen for further in vivo characterization:

Gpr12 siRNA2 sense strand

| | |
|---|---|
| GAGGCACGCCCAUCAGAUAUU; | SEQ ID NO:15 |

Gpr12 siRNA2 anti-sense strand

| | |
|---|---|
| UAUCUGAUGGGCGUGCCUCUU; | SEQ ID NO:16 |

non-targeting siRNA sense strand

| | |
|---|---|
| UAGCGACUAAACACAUCAAUU; | SEQ ID NO:17 |

non-targeting siRNA antisense strand

| | |
|---|---|
| UUGAUGUGUUUAGUCGCUAUU; | SEQ ID NO:18 |

### Example 6

### In Vivo Delivery of Synthetic Gpr12 siRNA in Mice

Animals and Environment. Young-adult (10-12 weeks old) C57BL/6 male mice (Taconic, N.Y.) were used. Upon arrival, mice were group-housed (5 mice) in standard laboratory cages and maintained on a 12:12 hours light-dark cycle. The experiments were always conducted during the light phase of the cycle. After surgery for cannulation, mice were single housed in individual cages and maintained so till the end of the experiment. With the exception of training and testing times, the mice had ad libitum access to food and water. Mice were maintained and bred under standard conditions, consistent with National Institutes of Health (NIH) guidelines and approved by the Institutional Animal Care and Use Committee.

Animal surgery and siRNA injection. For the injection of siRNA, mice were anesthetized with 20 mg/kg Avertin and implanted with a 33-gauge guide cannula bilateraly into the dorsal hippocampus (coordinates: A=−1.8 mm, L=+/−1.5 mm to a depth of 1.2 mm) or into amygdala (coordinates: A=−1.58 mm, L=+/−2.8 mm to a depth of 4.0 mm) (Franklin and Paxinos, 1997 The Mouse Brain in Stereotaxic Coordinates). Five to nine days after recovery from surgery, animals were injected with siRNA. siRNA was diluted to 0.5 μg per μl in 5% glucose and mixed with 6 equivalents of a 22 kDa linear polyethyleneimine (Fermentas). After 10 min of incubation at room temperature, 2 μl were injected into each hippocampus through an infusion cannula that was connected to a micro-syringe by a polyethylene tube. The entire infusion procedure took ˜2 min, and animals were handled gently to minimize stress. A total of 3 infusions of siRNA were given over a period of 3 days (1 μg siRNA per hippocampus per day).

siRNA mediated knockdown of Gpr12 may cause damage to the hippocampal formation. The hippocampal morphology of siRNA treated brains was evaluated.

siRNA injected animals were sacrificed one day after the behavioral experiments. Frozen brains were sliced into 15 μm sections and stained with Cresyl violet. Hippocampal morphology was evaluated on photographs of serial sections. For cannula verification, animals were injected with 1 μl of methyl blue dye and sacrificed immediately afterwards. Frozen brains were sliced into 15 μm sections. The position of the dye staining was determined microscopically and compared to (Franklin and Paxinos, 1997 The Mouse Brain in Stereotaxic Coordinates). Cannula verification was performed blind to the treatment of the subject.

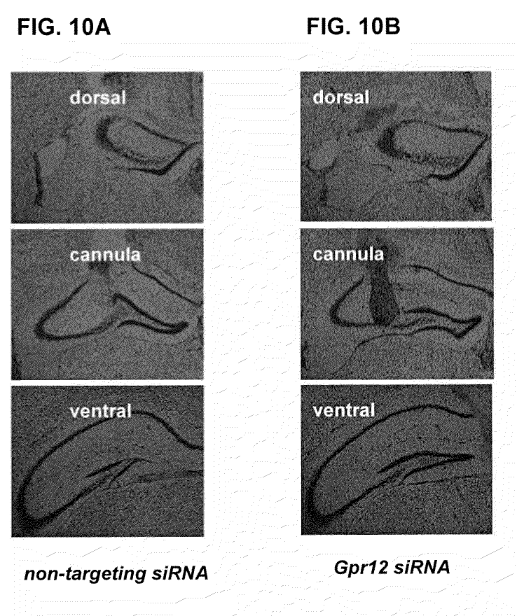

There were no obvious differences in hippocampal morphology between non-targeting siRNA (FIG. 10a) and Gpr12 siRNA treated mice (FIG. 10b). Hence, Gpr12 siRNA did not cause any obvious changes in brain morphology. Damage to the pyramidal cell layer was restricted to the area of cannulation. Note that the damage visible in FIG. 10 (middle panel) is facilitated by the removal of the hippocampal cannula. It does not represent the actual surgery induced alterations in hippocampal morphology, which is considered to be minimal and does not affect behavioral performance of the experimental subjects.

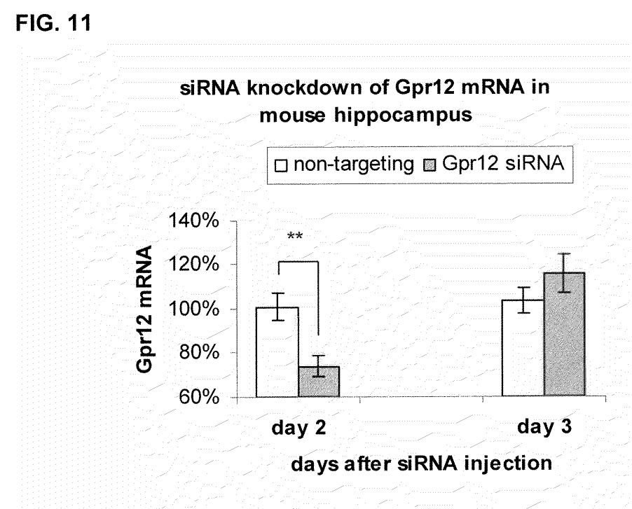

To confirm target knockdown by siRNA in vivo, we treated mice with intra-hippocampal siRNA for 3 days and determined Gpr12 mRNA levels at 2 and 3 days after the last siRNA infusion (FIG. 11).

For evaluation of gpr12 knockdown in vivo, siRNA injected hippocampal tissue of 6 mice per group was pooled. 6 individual RNA preparations were performed using the QIAgen RNeasy kit (Qiagen) according to the manufacturer's specifications. cDNA was generated using TaqMan Reverse transcriptase kit (Applied Biosystems). 2 real-time PCR reactions per RNA/cDNA replication were performed using the ABI prism and SDS 2.1 software. ABI assays on demand (Applied Biosystems) were used to test the mRNA levels of Gpr12. The average CT value for each cDNA sample was determined. Data was then normalized to TATA binding protein (TBP) and ΔCT values were determined. mRNA levels were normalized to a non-targeting control siRNA treated control group.

When compared to non-targeting control siRNA (n=6), Gpr12 siRNA (n=6) significantly reduced hippocampal mRNA levels of Gpr12 at 2 days after treatment (p<0.01). There was no significant effect of Gpr12 siRNA at 3 days after treatment, indicating that the Gpr12 mRNA knockdown was transient (p=0.25). These results confirm that siRNA reduced Gpr12 mRNA in hippocampus in vivo. However, target mRNA and protein levels may be affected differentially by Gpr12 siRNA. The actual protein levels of Gpr12 may be reduced to a stronger degree and for a longer time-span following siRNA treatment.

### Example 7

### Effect of siRNA Mediated Knockdown of Gpr12 on Contextual and Trace Conditioning

To assess contextual memory, a standardized contextual fear conditioning task originally developed for evaluation of memory in CREB knock-out mice was used ((Bourtchuladze et al., 1994 Cell 79, 59-68). On the training day, the mouse was placed into the conditioning chamber (Med Associates, Inc., VA) for 2 minutes before the onset of the unconditioned stimulus (US), a 0.5 mA foot shock of 2 seconds duration. For weak training (2 training trials), the US was repeated two times with a 1 min inter-trial interval between shocks. For strong training (5 training trials), 5 foot shocks were given with a 1 min inter-trial interval between shocks (Bourtchouladze et al., 1998 Learn Mem 5, 365-374.); (Scott et al., 2002 J Mol Neurosci 19, 171-177); (Tully et al., 2003 Nat Rev Drug Discov 2, 267-277). Training was performed using an automated software package (Med Associates, Inc., VA). After the last training trial, the mice were left in the conditioning chamber for another 30 sec and were then placed back in their home cages. Contextual memory was tested 24 hrs after training. The mouse was placed into the same training chamber and conditioning was assessed by scoring freezing behavior. Freezing was defined as the complete lack of movement in intervals of 5 seconds ((Fanselow and Bolles, 1979 J Comp Physiol Psychol 93, 736-744.); (Bourtchuladze et al., 1994 Cell 79, 59-68); (Bourtchouladze et al., 1998 Learn Mem 5, 365-374). Total testing time lasted 3 minutes. After each experimental subject, the experimental apparatus was thoroughly cleaned with 75% ethanol, water, dried, and ventilated. Each experiment was filmed. All experimenters were blind to the drug and training conditions.

All behavioral experiments were designed and performed in a balanced fashion, meaning that (i) for each experimental condition we used an equal number of experimental and control mice; (ii) each experimental condition was replicated several times, and replicate days were added to generate final number of subjects. The proceeding of each session was filmed. In each experiment, the experimenter was unaware (blind) to the treatment of the subjects during training and testing. Data were analyzed by Student's unpaired t test using a software package (StatView 5.0.1; SAS Institute, Inc). Except where stated, all values in the text and figures are expressed as MEAN±SEM.

Investigated first was the function of hippocampal Gpr12 in contextual memory. Mice were infused with non-targeting (n=19) or Gpr12 siRNA (n=20) into the hippocampus. 3 days after the last siRNA infusion the animals were trained with a contextual conditioning paradigm designed to induce a weak contextual memory (Scott et al., 2002 J Mol Neurosci 19, 171-177.), (Tully et al., 2003 Nat Rev Drug Discov 2, 267-277). Gpr12 DM-2 siRNA treated animals demonstrated significantly enhanced contextual memory at 24 h after training (24 h memory: p<0.05, FIG. 8a).

Next investigated was the function of Gpr12 in the amygdala for contextual memory formation. Mice were infused with non-targeting (n=20) or Gpr12 siRNA (n=21) into the amygdala and tested in contextual memory. As for Gpr12 knockdown in hippocampus, Gpr12 siRNA treated animals demonstrated significantly enhanced contextual memory at 24 h after training (24 h memory: p<0.01, FIG. 8b). Four mice (2× non-targeting siRNA, 2×Gpr12-2 siRNA) were excluded from the analysis because of inaccurate cannula placements.

For trace conditioning training a standardized mouse contextual fear conditioning equipment was used (Med Associates, Inc., VA; (Bourtchuladze et al., 1994 Cell 79, 59-68); (Bourtchouladze et al., 1998 Learn Mem 5, 365-374). On the training day, the mouse was placed into the conditioning chamber for 2 minutes before the onset of the conditioned stimulus (CS), a 2800 Hz tone, which lasted for 20 seconds at 75 dB. Sixty seconds after the end of the tone a 0.5 mA shock unconditioned stimulus (US) was delivered to the animal for two seconds. Previous experiments have revealed that this training paradigm induces poor trace fear memory in C57BL/6 mice, and that this memory can be facilitated by enhancers of the CREB pathway. After an additional 30 seconds in the chamber, the mouse was returned to its home cage. After each experimental subject, the experimental apparatus was thoroughly cleaned with 75% ethanol, water, dried, and ventilated for a few minutes.

Testing was done in a novel chamber located in another procedural room to avoid confounding effects of contextual conditioning. The internal conditioning chamber was removed and replaced with a mouse cage. Different colored tape was placed on the backside of each cage to differentiate one from another. Three different cages were used in rotation in order to decrease the possibility of scent contamination from subject to subject. A 30-watt lamp was placed inside the chamber to insure difference in illumination between training and testing. The cages were cleaned using a soapy solution instead of ethanol. Each test began with two minutes of light only (pre-CS), then 20 seconds of tone presentation (CS), followed by an additional 30 seconds of light only (post-CS). In the same manner as during training, the mice were scored one at a time for “freezing” in five-second intervals, as for contextual conditioning described above. The proceeding of each experiment was filmed. The proportion of the freezing response specific to the auditory memory was determined by subtraction of preCS freezing (non-specific) from CS freezing.

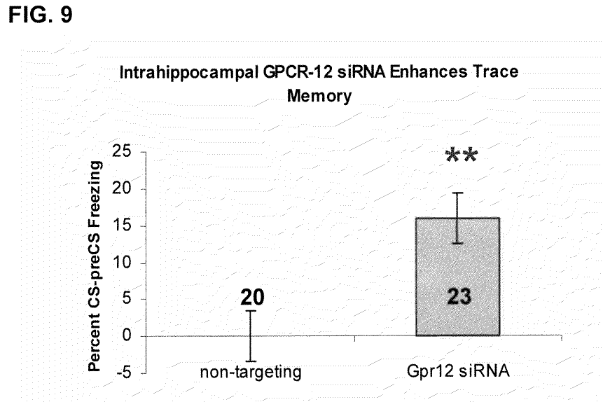

The function of hippocampal Gpr12 in trace fear memory was investigated. Mice were infused with non-targeting (n=20) or Gpr12 siRNA (n=23) into hippocampus as described for contextual conditioning. When trained with one CS/US pairing and a 60 seconds trace interval, Gpr12 DM-2 siRNA treated animals demonstrated significantly increased trace conditioning (CS-preCS: p<0.01, FIG. 9). Importantly, Gpr12 siRNA, but not control siRNA, treated animals increased their freezing response upon tone CS presentation. Thus, similarly to contextual conditioning, siRNA-mediated knockdown of hippocampal Gpr12 facilitated trace conditioning. Gpr12 siRNA did not significantly affect immediate freezing during trace fear conditioning (non-targeting siRNA: 3.3±1.5%; Gpr12 siRNA: 5.1±1.6%; p=0.44; data not shown).

Taken together these results strongly show that Gpr12 is a negative regulator of memory formation in both the hippocampus and the amygdala, two temporal lobe structures that are critical to memory formation in mice as well as in humans. Importantly, Gpr12 siRNA induced a ‘gain of function’ (that is, enhancement of memory formation). It is unlikely that this effect on behavioral plasticity is induced by side effects of Gpr12 siRNA. Thus we conclude that Gpr12 is a critical regulator of memory in hippocampus and amygdala.

All publications, patent and patent applications mentioned in this specification used herein to illuminate the background of the invention, and in particular, cases to provide additional details respecting the practice are incorporated herein by reference to the same extent as if each individual publication, patent or patent application was specifically and individually incorporated by reference.

While this invention has been particularly shown and described with references to preferred embodiments thereof, it will be understood by those skilled in the art that various changes in form and details may be made therein without departing from the scope of the invention encompassed by the appended claims.

1-35. (canceled)

36. A method comprising the steps of: (a) administering to an animal sufficient siRNA specific for a gene to inhibit said gene's function; (b) training said animal under conditions sufficient to induce transcription dependent memory formation in a normal untreated animal; and (c) determining the level of transcription dependent memory formation induced by the training of said treated animal.

37. The method of claim 36 wherein determination of an increase in transcription dependent memory formation in the treated animal relative to the transcription dependent memory formation in an untreated animal indicates that inhibition of the gene results in enhancement of transcription dependent memory formation.

38. The method of claim 36 wherein determination of a decrease in transcription dependent memory formation in the treated animal relative to the transcription dependent memory formation in an untreated animal indicates that inhibition of the gene results in inhibition of transcription dependent memory formation.

39. The method of claim 36 wherein said siRNA is administered before or simultaneously with the training session.

40. The method of claim 36 wherein the transcription dependent memory formation is long term memory formation.

41. The method of claim 36 wherein the transcription dependent memory formation is evidenced by performance of a specific cognitive task.

42. The method of claim 36 wherein said animal is a non-human mammal.

43. The method of claim 36 wherein step (b) training comprises multiple training sessions.

44. The method of claim 36 wherein step (b) training comprises a spaced training protocol.

45. The method of claim 36 wherein step (b) training comprises a contextual fear training protocol with single or multiple trials.

46. The method of claim 36 wherein step (b) training comprises trace fear conditioning with single or multiple trials.

47. The method of claim 36 wherein said training relates to a memory paradigm selected from the group consisting of contextual memory, temporal memory, spatial memory, episodic memory, passive avoidance memory, active avoidance memory, social transmission of food preferences memory, conditioned taste avoidance, and social recognition memory.

48. A method comprising the steps of: (a) administering to an animal sufficient siRNA specific for a gene to inhibit said gene's function; (b) training said animal under conditions sufficient to induce long term memory formation in a normal untreated animal; and (c) determining the level of long term memory formation induced by the training of said treated animal.

49. The method of claim 48 wherein determination of an increase in long term memory formation in the treated animal relative to the long term memory formation in an untreated animal indicates that inhibition of the gene results in enhancement of long term memory formation.

50. The method of claim 48 wherein determination of a decrease in long term memory formation in the treated animal relative to the long term memory formation in an untreated animal indicates that inhibition of the gene results in inhibition of long term memory formation.

51. The method of claim 48 wherein said siRNA is administered before or simultaneously with the training session.

52. The method of claim 48 wherein the long term memory formation is evidenced by performance of a specific cognitive task.

53. The method of claim 48 wherein said animal is a non-human mammal.

54. The method of claim 48 wherein step (b) training comprises multiple training sessions.

55. The method of claim 48 wherein step (b) training comprises a spaced training protocol.

56. The method of claim 48 wherein step (b) training comprises a contextual fear training protocol with single or multiple trials.

57. The method of claim 48 wherein step (b) training comprises trace fear conditioning with single or multiple trials.

58. The method of claim 48 wherein said training relates to a memory paradigm selected from the group consisting of contextual memory, temporal memory, spatial memory, episodic memory, passive avoidance memory, active avoidance memory, social transmission of food preferences memory, conditioned taste avoidance, and social recognition memory.

59. A method comprising the steps of: (a) administering to an animal sufficient si NA specific for a gene to inhibit said gene's function; (b) training said animal under conditions sufficient to produce an improvement in performance of a specific cognitive task in a normal untreated animal; and (c) determining the level of cognitive performance generated by training of said treated animal.

60. The method of claim 59 wherein determination of the level of cognitive performance in the treated animal relative to the level of cognitive performance in an untreated animal indicates that inhibition of the gene results in enhancement of cognitive performance.

61. The method of claim 59 wherein determination of a decrease in the level of cognitive performance in the treated animal relative to the level of cognitive performance in an untreated animal indicates that inhibition of the gene results in inhibition of cognitive performance.

62. The method of claim 59 wherein said siRNA is administered before or simultaneously with the training session.

63. The method of claim 59 wherein the cognitive performance is long term memory formation.

64. The method of claim 59 wherein the cognitive performance is evidenced by performance of a specific cognitive task.

65. The method of claim 59 wherein said animal is a non-human mammal.

66. The method of claim 59 wherein step (b) training comprises multiple training sessions.

67. The method of claim 59 wherein step (b) training comprises a spaced training protocol.

68. The method of claim 59 wherein step (b) training comprises a contextual fear training protocol with single or multiple trials.

69. The method of claim 59 wherein step (b) training comprises trace fear conditioning with single or multiple trials.

70. The method of claim 59 wherein said training relates to a memory paradigm selected from the group consisting of contextual memory, temporal memory, spatial memory, episodic memory, passive avoidance memory, active avoidance memory, social transmission of food preferences memory, conditioned taste avoidance, and social recognition memory.


SEQUENCE LISTING (18 sequences)

SEQ ID NO: 1 - RNA, 21 nt, Artificial Sequence
  siRNA sense strand control
uagcgacuaaacacaucaauu

SEQ ID NO: 2 - RNA, 21 nt, Artificial Sequence
  siRNA anti-sense strand control
uugauguguuuagucgcuauu

SEQ ID NO: 3 - RNA, 21 nt, Artificial Sequence
  CREB siRNA1 sense strand
caauacagcuggcuaacaauu

SEQ ID NO: 4 - RNA, 21 nt, Artificial Sequence
  CREB siRNA1 anti-sense strand
uuguuagccagcuguauuguu

SEQ ID NO: 5 - RNA, 21 nt, Artificial Sequence
  CREB siRNA2 sense strand
gcaagagaaugucguagaauu

SEQ ID NO: 6 - RNA, 21 nt, Artificial Sequence
  CREB siRNA2 anti-sense strand
uucuacgacauucucuugcuu

SEQ ID NO: 7 - RNA, 21 nt, Artificial Sequence
  PP1 sense strand
uagcgacuaaacacaucaauu

SEQ ID NO: 8 - RNA, 21 nt, Artificial Sequence
  PP1 anti-sense strand
uugauguguuuagucgcuauu

SEQ ID NO: 9 - protein, 334 aa, Mus musculus
MNEDPKVNLSGLPRDCIDAGAPENISAAVPSQGSVAESEPELVVNPWDIVLCSSGTLICCENAVVVLIIFHSPSLRAPMFLLIGSLALADLLAGLGLIINFVFAYLLQSEATKLVTIGLIVASFSASVCSLLAITVDRYLSLYYALTYHSERTVTFTYVMLVMLWGTSICLGLLPVMGWNCLRDESTCSVVRPLTKNNAAILSISFLFMFALMLQLYIQICKIVMRHAHQIALQHHFLATSHYVTTRKGVSTLALILGTFAACWMPFTLYSLIADYTYPSIYTYATLLPATYNSIINPVIYAFRNQEIQKALCLICCGCIPSSLSQRARSPSDV

SEQ ID NO: 10 - DNA, 2485 bp, Mus musculus
aagggaacaataatttgcagaccggccaactgcaatctaagagagggagtcgcttgctgttgtaagtctcctccgccagccctaacctgcttaccccgcattcctcctgttcatcccgaaaacccggccgtttacaattctttaggggaaagcataagaagccgagccccagggtcaagggcgcctcggggaagccacaggatcaaagtaggtcgccagactctccggccgttcgagtgggtcttcgcatgactgttgcaggcgggcgtccacggtggcgggctcccgcccctcacgcagctgcgacctgcgggggcgcgcgcagcctcgtggggttcccgcggatgcgcgcccggcggggagcgcggagggcggagagccgggcgcgagcaccgcagctcacctgccgcgggcgccaccacggacgtgccacgcgggtggcccgagctattcggcagcactgaaggagccacccctcggccagggcgtgccaaggacaggggttaaaatgaacgaagacccgaaggtcaatttaagcgggctgcctcgggactgtatagatgccggtgctccagagaacatctcagccgctgtcccctcccagggctctgttgcggagtcagaacccgagctcgttgtcaacccctgggacattgtcttgtgcagctcaggaaccctcatctgctgtgaaaatgccgttgtggtccttatcatcttccacagccccagcctgcgagcccccatgttcctactgataggcagcctggctcttgcagacctgctggctggcctgggactcatcatcaattttgtttttgcgtacctgcttcagtcagaagccaccaagctggtcaccatcggactcattgtcgcctctttctctgcctctgtctgcagtttgctggctattactgtggaccgctacctctcgctatattacgccctgacgtaccactccgagaggaccgtcacctttacctatgtcatgctagtgatgctctggggaacctccatctgcctggggctgctgcccgtcatgggctggaactgcttgagggacgagtccacctgcagcgtggtcagacctctcactaagaacaacgctgccatcctctccatctccttcctcttcatgtttgctctgatgcttcagctctacatccagatttgtaagattgtgatgaggcacgcccatcagatagccctgcagcaccacttcctggctacatcgcactatgtgactacccggaaaggggtctcgaccctggctctcatcctagggacctttgctgcctgctggatgcctttcaccctctattccttgatcgccgattacacctacccttcgatctatacctatgccaccctcctgcccgccacctacaattccatcatcaaccctgtcatttacgctttcagaaaccaagagatccagaaagccctctgcctcatttgctgtgggtgcatcccttcctcgctgtctcagagagctcggtctcccagcgatgtgtagcagccttctcctcataggacgctgcctctaccaagcgctcccacctcccagggcggccagtgatttccttccttaaattctttgcactggatctcacaagcagaagcaatgacatcttttagacacgtattgacagtggaaatcatcttaccagtgttttttaaaaaaaaaacaaaacaaaactcgacttctcggctcagcattctgttgtttggtttgggagttaggatttgtttgtttgtttgcttgtttgtttgtttggagggtgtaatgggacctcatgtggccatgaaattatacaaaagtctcgggattttttaacctaggcttgaaaataaatcaaagttttaaaggaaactggagaaggaaatactttttctgaaggaaatacttttttttttttaatcaaggtagatcttccattctgtatgtatctaacaggataggagctttgccatataaccaaaatagtttatataattacatttggaagggcttgtgtttatttctaggaattcagtaataagtgaccagtaacagaggcgcgaactcctttctttcctttcagcagtagtgactgctcttaagaatcactttgcagtttctctgtgttacagtttggtatgcatggttacctgtggtagtcagatcactaattgcaatattgccatgttaaacccagaattaaaagagtcattttttcttcaatacagtttttgaaatatcctttccaaagtgagtcatgaaaaaaatgtttccaattacatatgagatagcactggttagatttgtcattgtgatttttaaaactctagactggtggttttcagaaaacaaaagagaaaatattaacagcatctattgaaagaagattttatttatttttaatatattctgagagaataaatggtgtgatactattaagaaatatacaaacatgacttttcaaatctctaaaaaaaaaaaaaaaaa

SEQ ID NO: 11 - DNA, 2271 bp, Mus musculus
cggcatgggagatgcaattagccaatgtcggttttcagcgttttggcaagtgtgcgagtgtgcatgtgccgcctcgggagtcctgatccgtgtttccctcagagacaaacagcatttcggttgcagactttagcttttgtttttaattcctgaagctcgtggcattttgacactgatagctgagcccagggttgtctgtctttctctgtgtgttttgcatgatcttggattggcaccctactgtacccaaacattaaaaagcctgtctttccgttgaagaggacaggggttaaaatgaacgaagacccgaaggtcaatttaagcgggctgcctcgggactgtatagatgccggtgctccagagaacatctcagccgctgtcccctcccagggctctgttgcggagtcagaacccgagctcgttgtcaacccctgggacattgtcttgtgcagctcaggaaccctcatctgctgtgaaaatgccgttgtggtccttatcatcttccacagccccagcctgcgagcccccatgttcctactgataggcagcctggctcttgcagacctgctggctggcctgggactcatcatcaattttgtttttgcgtacctgcttcagtcagaagccaccaagctggtcaccatcggactcattgtcgcctctttctctgcctctgtctgcagtttgctggctattactgtggaccgctacctctcgctatattacgccctgacgtaccactccgagaggaccgtcacctttacctatgtcatgctagtgatgctctggggaacctccatctgcctggggctgctgcccgtcatgggctggaactgcttgagggacgagtccacctgcagcgtggtcagacctctcactaagaacaacgctgccatcctctccatctccttcctcttcatgtttgctctgatgcttcagctctacatccagatttgtaagattgtgatgaggcacgcccatcagatagccctgcagcaccacttcctggctacatcgcactatgtgactacccggaaaggggtctcgaccctggctctcatcctagggacctttgctgcctgctggatgcctttcaccctctattccttgatcgccgattacacctacccttcgatctatacctatgccaccctcctgcccgccacctacaattccatcatcaaccctgtcatttacgctttcagaaaccaagagatccagaaagccctctgcctcatttgctgtgggtgcatcccttcctcgctgtctcagagagctcggtctcccagcgatgtgtagcagccttctcctcataggacgctgcctctaccaagcgctcccacctcccagggcggccagtgatttccttccttaaattctttgcactggatctcacaagcagaagcaatgacatcttttagacacgtattgacagtggaaatcatcttaccagtgttttttaaaaaaaaaacaaaacaaaactcgacttctcggctcagcattctgttgtttggtttgggagttaggatttgtttgtttgtttgcttgtttgtttgtttggagggtgtaatgggacctcatgtggccatgaaattatacaaaagtctcgggattttttaacctaggcttgaaaataaatcaaagttttaaaggaaactggagaaggaaatactttttctgaaggaaatacttttttttttttaatcaaggtagatcttccattctgtatgtatctaacaggataggagctttgccatataaccaaaatagtttatataattacatttggaagggcttgtgtttatttctaggaattcagtaataagtgaccagtaacagaggcgcgaactcctttctttcctttcagcagtagtgactgctcttaagaatcactttgcagtttctctgtgttacagtttggtatgcatggttacctgtggtagtcagatcactaattgcaatattgccatgttaaacccagaattaaaagagtcattttttcttcaatacagtttttgaaatatcctttccaaagtgagtcatgaaaaaaatgtttccaattacatatgagatagcactggttagatttgtcattgtgatttttaaaactctagactggtggttttcagaaaacaaaagagaaaatattaacagcatctattgaaagaagattttatttatttttaatatattctgagagaataaatggtgtgatactattaagaaatatacaaacatgacttttcaaatctctaaaaaaaaaaaaaaaaa

SEQ ID NO: 12 - protein, 334 aa, Mus musculus; identical to SEQ ID NO: 9

SEQ ID NO: 13 - DNA, 1005 bp, Homo sapiens
atgaatgaagacctgaaggtcaatttaagcgggctgcctcgggattatttagatgccgctgctgcggagaacatctcggctgctgtctcctcccgggttcctgccgtagagccagagcctgagctcgtagtcaacccctgggacattgtcttgtgtacctcgggaaccctcatctcctgtgaaaatgccattgtggtccttatcatcttccacaaccccagcctgcgagcacccatgttcctgctaataggcagcctggctcttgcagacctgctggccggcattggactcatcaccaattttgtttttgcctacctgcttcagtcagaagccaccaagctggtcacgatcggcctcattgtcgcctctttctctgcctctgtctgcagcttgctggctatcactgttgaccgctacctctcactgtactacgctctgacgtaccattcggagaggacggtcacgtttacctatgtcatgctcgtcatgctctgggggacctccatctgcctggggctgctgcccgtcatgggctggaactgcctccgagacgagtccacctgcagcgtggtcagaccgctcaccaagaacaacgcggccatcctctcggtgtccttcctcttcatgtttgcgctcatgcttcagctctacatccagatctgtaagattgtgatgaggcacgcccatcagatagccctgcagcaccacttcctggccacgtcgcactatgtgaccacccggaaaggggtctccaccctggctatcatcctggggacgtttgctgcttgctggatgcctttcaccctctattccttgatagcggattacacctacccctccatctatacctacgccaccctcctgcccgccacctacaattccatcatcaaccctgtcatatatgctttcagaaaccaagagatccagaaagcgctctgtctcatttgctgcggctgcatcccgtccagtctcgcccagagagcgcgctcgcccagtgatgtgtag

SEQ ID NO: 14 - protein, 334 aa, Homo sapiens
MNEDLKVNLSGLPRDYLDAAAAENISAAVSSRVPAVEPEPELVVNPWDIVLCTSGTLISCENAIVVLIIFHNPSLRAPMFLLIGSLALADLLAGIGLITNFVFAYLLQSEATKLVTIGLIVASFSASVCSLLAITVDRYLSLYYALTYHSERTVTFTYVMLVMLWGTSICLGLLPVMGWNCLRDESTCSVVRPLTKNNAAILSVSFLFMFALMLQLYIQICKIVMRHAHQIALQHHFLATSHYVTTRKGVSTLAIILGTFAACWMPFTLYSLIADYTYPSIYTYATLLPATYNSIINPVIYAFRNQEIQKALCLICCGCIPSSLAQRARSPSDV

SEQ ID NO: 15 - RNA, 21 nt, Artificial Sequence
  Gpr12 siRNA2 sense strand
gaggcacgcccaucagauauu

SEQ ID NO: 16 - RNA, 21 nt, Artificial Sequence
  Gpr12 siRNA2  anti-sense strand
uaucugaugggcgugccucuu

SEQ ID NO: 17 - RNA, 21 nt, Artificial Sequence
  non-targeting siRNA sense strand
uagcgacuaaacacaucaauu

SEQ ID NO: 18 - RNA, 21 nt, Artificial Sequence
  non-targeting siRNA antisense strand
uugauguguuuagucgcuauu